(19) United States
(12) Patent Application Publication
(10) Pub. No.: US 2010/0041107 A1
(43) Pub. Date: Feb. 18, 2010

(54) METHOD OF REDUCING GENE EXPRESSION USING MODIFIED CODON USAGE

(75) Inventors: Andrea Herold, Ketsch (DE); Corinna Klopprogge, Mannheim (DE); Hartwig Schröder, Nussloch (DE); Osker Zelder, Speyer (DE); Weol Kyu Jeong, Hirschberg (DE)

Correspondence Address: CONNOLLY BOVE LODGE & HUTZ, LLP, P O BOX 2207, WILMINGTON, DE, 19899, US

(73) Assignee: BASF SE, Ludwigshafen (DE)

(21) Appl. No.: 12/446,767

(22) PCT Filed: Oct. 18, 2007
(86) PCT No.: PCT/EP2007/061151

§ 371 Date: Apr. 23, 2009

(30) Foreign Application Priority Data
Oct. 24, 2006 (EP) 06122882.1

(51) Int. Cl. C12P 13/12; C12P 1/00; C12P 13/04; C12P 13/08; C12P 7/64; C12N 5/10; C12N 5/04; C12N 1/21; C12N 15/00; C12N 15/11

(52) U.S. Cl. 435/113; 435/41; 435/106; 435/115; 435/134; 435/348; 435/419; 435/252.32; 435/320.1; 536/23.1

(57) ABSTRACT

The present invention is directed to a method of reducing the amount of at least one polypeptide in a host cell by expressing a nucleotide sequence encoding for the polypeptide in the host cell wherein the nucleotide sequence uses codons that are rarely used according to the codon usage of the host organism. Furthermore, the present invention relates to nucleotide sequences encoding for a polypeptide with a codon usage that has been adjusted to use codons that are only rarely used according to the codon usage of the host organism. The present invention further relates to the use of such sequences and methods for producing fine chemicals such as amino acids, sugars, lipids, oils, carbohydrates, vitamins, cofactors etc.

### OBJECT OF THE INVENTION

The present invention is directed to a method of reducing the amount of at least one polypeptide in a host cell by expressing in the host cell a nucleotide sequence encoding for a polypeptide wherein the nucleotide sequence uses codons that are rarely used according to the codon usage of the host organism.

Furthermore, the present invention relates to nucleotide sequences encoding for a polypeptide with a codon usage that has been adjusted to use codons that are only rarely used according to the codon usage of the host organism.

The present invention further relates to the use of such sequences and methods for producing fine chemicals such as amino acids, sugars, lipids, oils, carbohydrates, vitamins, cofactors etc.

### BACKGROUND

In a lot of biotechnological processes it is necessary to modulate gene expression. Thus, for some applications it is necessary to increase the expression of a certain gene product and to thereby increase the amount and/or activity of e.g. a protein in the host cell in which, the gene of interest is (over) expressed. Similarly, it may be desirable to reduce the amount of expression of an endogenous gene in a host cell. Furthermore, it may be desireable to fine-tune the expression level of endogenous or heterologous genes.

The fermentative production of so-called fine chemicals is today typically carried out in microorganisms such as Corynebacterium glutamicum (C. glutamicum), Escherichia coli (E. coli), Saccharomyces cerevisiae (S. cerevisiae), Schizzosaccharomycs pombe (S. pombe), Pichia pastoris (P. pastoris), Aspergillus niger, Bacillus subtilis, Ashbya gossypii or Gluconobacter oxydans.

Fine chemicals which include e.g. organic acids such as lactic acid, proteogenic or non-proteogenic amino acids, purine and pyrimidine bases, carbohydrates, aromatic compounds, vitamins and cofactors, lipids, saturated and unsaturated fatty acids are typically used and needed in the pharmaceutical, agriculture, cosmetic as well as food and feed industry.

As regards for example the amino acid methionine, currently worldwide annual production amounts to about 500,000 tons. The current, industrial production process is not by fermentation but a multi-step chemical process. Methionine is the first limiting amino acid in livestock of poultry feed and due to this mainly applied as a feed supplement. Various attempts have been published in the prior art to produce methionine e.g. using microorganisms such as E. coli.

Other amino acids such as glutamate, lysine, threonine and threonine, are produced by e.g. fermentation methods. For these purposes, certain microorganisms such as C. glutamicum have been proven to be particularly suited. The production of amino acids by fermentation has the particular advantage that only L-amino acids are produced and that environmentally problematic chemicals such as solvents as they are typically used in chemical synthesis are avoided.

Some of the attempts in the prior art to produce fine chemicals such as amino acids, lipids, vitamins or carbohydrates in microorganisms such as E. coli and C. glutamicum have tried to achieve this goal by e.g. increasing the expression of genes involved in the biosynthetic pathways of the respective fine chemicals. If e.g. a certain step in the biosynthetic pathway of an amino acid such as methionine or lysine is known to be rate-limiting, over-expression of the respective enzyme may allow obtaining a microorganism that yields more product of the catalysed reaction and therefore will ultimately lead to an enhanced production of the respective amino acid. Similarly, if a certain enzymatic step in the biosynthetic pathway of an e.g. desired amino acid is known to be non-desirable as it channels a lot of metabolic energy into formation of undesired by-products it may be contemplated to down-regulate expression of the respective enzymatic activity in order to favour only such metabolic reactions that ultimately lead to the formation of the amino acid in question.

Attempts to increase production of e.g. methionine and lysine by up-and/or down regulating the expression of genes being involved in the biosynthetic pathway of methionine or lysine production are e.g. described in WO 02/10209, WO 2006008097, or WO2005059093.

Typically, overexpression of a certain gene in a microorganism such as E. coli or C. glutamicum or other host cells such as P. pastoris, A. niger or even mammalian cell culture systems may be achieved by transforming the respective cell with a vector that comprises a nucleotide sequence encoding for the desired protein and which further comprises elements that allow the vector to drive expression of the nucleotide sequence encoding, e.g. for a certain enzyme. Using this approach foreign proteins, i.e. proteins that are encoded by sequences that are not naturally found in the host cell that is used for expression as well as endogenous host cell-specific proteins may be overexpressed. Other typical methods include increasing of copy number of the respective genes in the chromosome, inserting strong promoters for regulating the transcription of the chromosomal copy of the respective genes and enhancing translational initiation by optimization of the ribosomal binding site (RBS).

For down-regulating expression of certain factors within e.g. microorganisms, a multitude of technologies such as gene knockout approaches, antisense technology, RNAi technology etc. are available. Some of the technologies for down-regulation of genes lead to a complete loss-of-function for the respective factors due to the absence of e.g. any enzyme being produced. This can be problematic where only a certain degree of reduction in protein amount is the overall goal. In such cases, one may delete the wild type copy of the respective gene and replace it with a mutant version that shows decreased activity or express it from a weak promoter. Such approaches are however rather cumbersome and other means of reducing partially expression of e.g. a protein without changing its amino acid sequence are highly desirable not only for producing fine chemicals in e.g. microorganisms, but also for other purposes.

In view of this situation, it is one object of the present invention to provide methods and nucleotide sequences encoding for polypeptides which can be used to reduce expression of polypeptides in host cells with a particular focus on industrially important microorganisms such as C. glutamicum. It is a further object to provide such methods and sequences for producing fine chemicals.

These and other objectives as they will become apparent from the ensuing description of the invention are solved by the present invention as described in the independent claims. The dependent claims relate to preferred embodiments.

### SUMMARY OF THE INVENTION

In one embodiment the invention relates to a method of reducing the amount of at least one polypeptide in a host cell. This method of reducing the amount of a polypeptide comprises the step of expressing in the host cell a nucleotide sequence which encodes for said polypeptide instead of the endogenous nucleotide sequence which encodes for a polypeptide of substantially the same amino acid sequence and/or function. This nucleotide sequence is derived from the endogenous nucleotide sequence such that codons of the endogenous nucleotide sequence are exchanged with less frequently used and preferably the least frequently used codons according to the codon usage of the host organism. In a preferred embodiment the reference codon usage will be determined on the basis of the group of abundant proteins of the host cell. The nucleotide sequence which: will thus have been optimised for reduced expression of a polypeptide may also be designated as modified nucleotide sequence while the endogenous sequence may be designated as starling or non-modified nucleotide sequence.

In a preferred embodiment frequent, very frequent or extremely frequent codons are exchanged for rare, preferably for very rare and most preferably for extremely rare codons. The reference codon usage will be based on the codon usage of the host, organism and preferably on the codon usage of abundant proteins of the host organism.

This method of reducing the amount of a polypeptide thus comprises the step of expressing in the host cell a modified nucleotide sequence which encodes for said polypeptide instead of the endogenous nucleotide sequence which encodes for a polypeptide of substantially the same amino acid sequence and/or function. The modified nucleotide sequence is derived from the (non-modified) endogenous nucleotide sequence such that codons of the non-modified nucleotide sequence, are exchanged in the modified nucleotide sequence with less frequently used and preferably the least frequently used codons. The reference codon usage will be based in the codon usage of the host organism and preferably on the codon usage of abundant proteins of the host organism, in a preferred embodiment frequent, very frequent or extremely frequent codons are thus exchanged for rare, very rare or extremely rare codons. In a particularly preferred embodiment, one, some or all codons are replaced by the least frequently used codons.

In one embodiment at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequences may be replaced in die modified nucleotide sequence by less frequently used codons for the respective amino acid. In an even more preferred embodiment the afore-mentioned number of codons to be replaced refers to frequent, very frequent or extremely frequent codons. In a particularly preferred embodiment these codons are replaced by rare, preferably very rare and most preferably extremely rare codons. In another particularly preferred embodiment, the above numbers of codons are replaced by the least frequently used codons respectively. In all these case is the reference codon usage based in the codon usage of the host organism and preferably on the codon usage of abundant proteins of the host cell.

In a preferred embodiment one will use modified nucleotide sequence which uses only rare, very rare, extremely rare or preferably the least frequently used codons for each replaced amino acid as determined for the group of abundant proteins of the host cell.

The polypeptides that are to be expressed by this method of reducing the amount of a polypeptide in a host cell are endogenous polypeptides with the proviso that the modified nucleotide sequence must not be identical with the starting nucleotide sequences of the respective polypeptides of the host cell.

The host, cells may be selected from microorganisms, insect cells, plant cells or mammalian cell culture systems.

Using this method, expression of the respective polypeptide may he reduced in the host cell by at least 5%, 10%, 20%, 30%, 40%, 50%, 60%, 70%, 80%, 90% or 95%. The extent of reduction of expression is determined in comparison to the level of expression of the endogenous polypeptide that is expressed from the endogenous non-modified nucleotide sequence under comparable conditions.

A preferred embodiment of this method of reducing the amount of a polypeptide in a host cell relates to methods wherein the amount of a polypeptide being expressed in Corynebacterium and particularly preferably in C. glutamicum is reduced.

Such a method comprises the step expressing in Corynebacterium and preferably in C. glutamicum a modified nucleotide sequence encoding for a polypeptide instead of the endogenous nucleotide sequence encoding for the respective polypeptide of substantially the same amino acid sequence and/or function. In one embodiment at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably ail of the codons of the non-modified nucleotide sequences may be replaced in the modified nucleotide sequence by less frequently used codons for the respective amino acid. In an even more preferred embodiment the afore-mentioned number of codons to be replaced refers to frequent, very frequent or extremely frequent codons. In a particularly preferred embodiment these codons are replaced by rare, preferably very rare and most preferably extremely rare codons. In another particularly preferred embodiment, the above numbers of codons are replaced by the least frequently used codons respectively. In all these cases will the reference codon usage based on the codon usage of Corynebacterium and preferably C. glutamicum. Preferably the reference codon usage will be based on the codon usage of abundant proteins of Corynebacterium and preferably C. glutamicum.

For reducing expression of polypeptides in the genus of Corynebacterium and particularly in the species of C. glutamicum based on altered codon usage, in another embodiment the non-modified nucleotide sequence encoding for the polypeptide may be modified such that at least one, at least two, at least three, at least four, at least five, at least six, at least seven, at least, eight, at least nine, at least ten, preferably at least 1%, at least 2%, at least 4%, at least 6%, at least 8%, at least 10%, more preferably at least 20%, at least 40%, at least 60%, at least 80%, even more preferably at least. 90% or least 95%. and most preferably all of the codons of the non-modified nucleotide sequence are replaced in the resulting modified nucleotide sequence by less frequently used codons for the respective amino acid according to Table 1 and preferably according to Table 2. In an even more preferred embodiment the afore-mentioned number of codons to be replaced refers to frequent, very frequent and extremely frequent codons. In another particularly preferred embodiment, tire above numbers of codons are replaced by one of the two least frequently used codons encoding lor the respective amino acid(s) as set forth in Table 1 and preferably in Table 2.

In another embodiment of the invention which relates to a method of decreasing the amount of polypeptide in Corynebacterium and particularly preferably in C. glutamicum, said modified nucleotide sequence uses the codons ACG for threonine, GAT for aspartic acid, GAA for glutamic acid, AGA and AGG for arginine and/or TTG for the start codon encoding methionine.

In yet another embodiment of the invention which relates to a method of decreasing the amount of polypeptide in Corynebacterium and particularly preferably in C. glutamicum, at least one codon of the modified nucleotide sequences may be selected from the codon usage of Table 3 and preferably of Table 4.

Thus, another particularly preferred embodiment of the invention relates to methods of decreasing the amount of a polypeptide in Corynebacterium and particularly preferred in C. glutamicum wherein at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequence are replaced in the resulting modified nucleotide sequence by the codons for the respective amino acid according to Table 3 and preferably according to Table 4. in an even more preferred embodiment the afore-mentioned number of codons to be replaced refers to frequent, very frequent, extremely frequent or the most frequent codons.

Another particularly preferred embodiment of the invention relate to methods of decreasing the amount of a polypeptide in Cornebacterium and particularly preferred in C. glutamicum wherein all codons of the non-modified nucleotide sequence are replaced in the resulting modified nucleotide sequence by the codons for the respective amino acid according to Table 3 and preferably according to Table 4.

One aspect of the invention also relates to modified nucleotide sequences as they may be used in the above-described methods of reducing the amount of a polypeptide in a host organism.

Such modified nucleotide sequences are derived from the endogenous nucleotide sequences encoding for a polypeptide of substantially the same amino acid sequence and/or function with the codon usage of the modified nucleotide sequence being adjusted such that codons of the non-modified (wild-type) sequences are replaced by less frequently used codons. The reference codon usage will be based on the codon usage of the host organism and preferably on the codon usage of abundant proteins of the host organism.

Of course, the invention in a preferred embodiment relates to such modified nucleotide sequences that have been derived for a specific polypeptide by replacing at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at. least 40%, at least 60%, at least 80%, even more preferably at least 90% or least 95% and most preferably all of the codons of the non-modified, nucleotide sequences are replaced in the modified nucleotide sequence by less frequently used codons for the respective amino acid. In an even more preferred embodiment the afore-mentioned number of codons to be replaced refers to frequent, very frequent, extremely frequent or the most frequent codons. In another particularly preferred embodiment, the above numbers of codons are replaced by the least frequently used codons. In all cases will the reference codon usage be based on the codon usage of the host organism and preferably on the codon usage of abundant proteins of the host organism.

In one preferred embodiment the modified nucleotide sequences will use for each replaced amino acid the least frequently used, codon according to the codon usage of the host cell and preferably according to the codon usage of the abundant proteins of the host cell.

In case of modified nucleotide sequences that are to be expressed in Corynebacterium and particularly preferably in C. glutamicum for reducing the amount of the respective encoded polypeptide, at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequences may be replaced in the modified nucleotide sequence by less frequently used codons for the respective amino acid. In an even more preferred embodiment the afore-mentioned number of codons to be replaced refers to frequent, very frequent, extremely frequent or the most frequent codons. In another particularly preferred embodiment, the above number of codons are replaced by the least frequently used codons. In all these cases will the reference codon usage based on the codon usage of the Corynebacterium and preferably C. glutamicum and preferably on the codon usage of abundant proteins of Corynebacterium and preferably C. glutamicum.

In ease of modified nucleotide sequences that are to be expressed in Corynebacterium and particularly preferably in C. glutamicum for reducing the amount of the respective encoded polypeptide, at least one, at least two, at least three, at least four, at least, five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequence are replaced in the resulting modified nucleotide sequence by less frequently used codons for the respective amino acid according to Table 1 and preferably according to Table 2. In an even more preferred embodiment the afore-mentioned number of codons to be replaced refers to frequent, very frequent, extremely frequent or the most frequent codons. In another particularly preferred embodiment, the afore-mentioned numbers of codons are replaced by one of the two least frequently used codons encoding for the respective amino acid(s) as set forth in Table 1 and preferably in Table 2.

In another embodiment the modified nucleotide sequence which is used for reducing expression of a polypeptide in Corynebacterium and particularly preferably in C. glutamicum uses the codons ACG for threonine, GAT for aspartic acid, GAA for glutamic acid, AGA and AGO for arginine and/or TTG for the start codon encoding methionine.

In yet another embodiment of the invention the modified nucleotide sequence which is used for reducing expression of a polypeptide in Corynebacterium and particularly preferably in C. glutamicum may comprise at least one codon being selected from Table 3 and preferably from Table 4.

Thus, another particularly preferred embodiment of the invention relates to modified nucleotide sequences for reducing expression of a polypeptide in Corynebacterium and particularly preferred in C. glutamicum wherein at least one, at least two, at least three, at least four, at least live, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequence are replaced in the resulting modified nucleotide sequence by the codons for the respective amino acid according to Table 3 and preferably according to Table 4. In an even more preferred embodiment the afore-mentioned number of codons to be replaced refers to frequent, very frequent, extremely frequent and most frequent codons.

Another particularly preferred embodiment of the invention relates to modified nucleotide sequences for reducing expression of a polypeptide in Corynebacterium and particularly preferably in C. glutamicum wherein ail codons of the non-modified nucleotide sequence are replaced in the resulting modified nucleotide sequence by the codons for the respective amino acid according to Table 3 and preferably according to Table 4.

In the situation where they are used for reducing expression of polypeptides in host cells and preferably in C. glutamicum these modified nucleotide sequences will preferably be selected from the group comprising nucleotide sequences encoding genes of biosynthetic pathways of fine chemicals such as amino acids, sugars, carbohydrates, lipids, oils, vitamins, cofactors etc. if down-regulation of such genes is known to favour production of the fine chemical(s). They may particularly be selected from the group comprising the sequences coding for genes of biosynthetic pathways being involved in the synthesis of amino acids such as glycine, lysine, cysteine, tryptophane or methionine.

A particularly preferred embodiment of the invention relates to a method of decreasing the expression of isocitrate dehydrogenase in a microorganism by adapting the codon usage as described herein. The microorganism can be a Corynebacterium, with C. glutamicum being preferred. These methods may be used improve synthesis of amino acids and particularly of methionine and/or lysine.

A host cell which comprises a modified nucleotide sequence that will lead to a reduced expression of a polypeptide in the host cell as described above also forms part of the invention. Such a host cell, which may be C. glutamicum will preferably comprise a modified nucleotide sequence that will lead to a reduced expression of isocitrate dehydrogenase

The present invention also relates to the use of the aforementioned modified nucleotide sequences and/or host cells for producing fine chemicals such as amino acids, sugars, lipids, oils, vitamins, cofactors, carbohydrates etc. They may be particularly used for production of amino acids such as glycine, lysine, threonine, cysteine or methionine.

### FIGURES

FIG. 1 shows the wild type sequence of isocitrate dehydrogenase (SEQ ID No. 1)

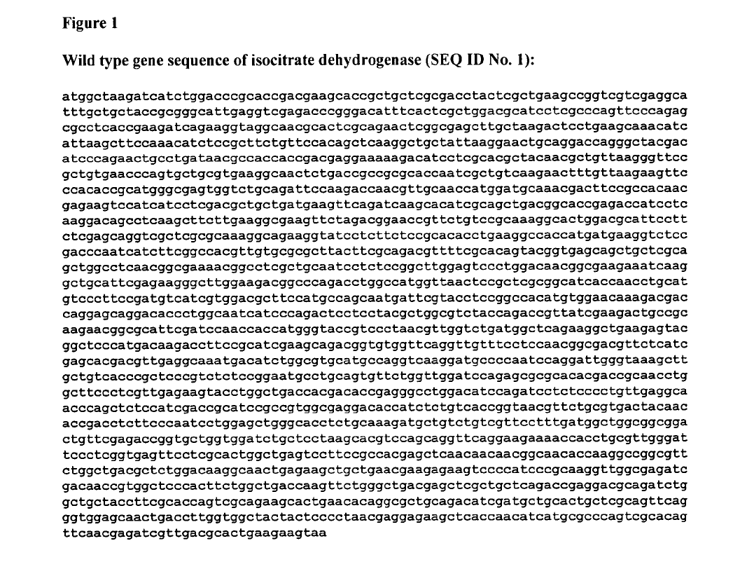

FIG. 2a) shows the codon usage amended isocitrate dehydrogenase (led) carrying an ATG-GTG mutation (SEQ ID No. 2). The mutation is shadowed in grey. FIG. 2b) shows the vector insert that was used to replace the endogenous gene (SEQ ID No. 3). The mutation is shadowed in grey. The restriction sites are underlined.

FIG. 3a) shows the codon usage amended isocitrate dehydrogenase (led) CA2 (SEQ ID No. 4). The mutation, is shadowed in grey. FIG. 3b) shows the vector insert that was used to replace the endogenous gene (SEQ ID No. 5). The mutation is shadowed in grey. The restriction sites are underlined.

### DETAILED DESCRIPTION OF THE INVENTION

As has been set out in the introductory part., it can be desirable in some case to overexpress e.g. foreign genes in a certain host cell as this approach allows to confer novel and unique characteristics to a host cell if e.g. a gene encoding for a certain enzymatic activity is introduced which naturally is not found in the host cell.

However, overexpression of foreign genes having no counterpart in the host ail by using e.g. expression vectors such as plasmids has encountered problems. The same has been observed for overexpression of genes which have a counterpart in the host organism as regards their function but which use a nucleotide sequence that is typically not found within the host organism. The failure of host cells such, as E. coli or C. glutamicum to express certain foreign (heterologous) sequences may be due to altered codon usage (see e.g. WO 2004/042059).

The genetic code is degenerate which, means that a certain amino acid may be encoded by a number of different base triplets. Codon usage refers to the observation that a certain organism will typically not use every possible codon for a certain amino acid with the same frequency. Instead an organism will typically show certain preferences, i.e. a bias for specific codons meaning that these codons are found more frequently in the transcribed genes of an organism.

However, codon usage manipulation has been used in the past for increasing expression of foreign genes. For reducing expression of genes, most commonly a part or the complete coding region has been removed, from the genome (so called “knock out” approach). Similarly, mutants with reduced activity or the modulation of transcriptional activity e.g. by weak promoters have been applied in this context.

The present invention relies partly on the surprising finding that, if codons of a nucleotide sequence encoding for an endogenous polypeptide are exchanged against codons which are less frequently found and used in the host cell, one will observe a reduced expression level for this polypeptide due to the amended codon usage.

The use of codons which are less frequently used in the host organism for reducing expression of a polypeptide has numerous advantages. As the modified nucleotide sequence can be integrated into the native genomic locus, the genomic integrity of the organism will be largely preserved. Moreover depending on the number of codons that are exchanged and the frequency of the codons that are introduced it may be possible to fine tune the reduction of the specific factor.

The present invention relies further on the surprising finding that determination of the codon usage of an organism may give different results depending on whether the codon usage is determined for abundant proteins of the host cell or for the host organism as a whole.

Typically, codon usage tables in the prior art for organisms such as E. coli etc. have been based on an analysis of the complete genome. The inventors of the present invention have found for the case of C. glutamicum that codon usage analysis of abundant proteins will give quite different results compared to codon usage frequencies as determined for the complete organism of C. glutamicum. Without being wanted to be bound to a theory, it is assumed that the specific codon usage frequency of abundant proteins in an organism such as C. glutamicum reflects certain requirements as to the codon composition of a highly expressed nucleotide sequence.

The specific codon usage distribution of highly expressed genes may e.g. reflect preferences for codons that are recognised by tRNAs that are also frequently and abundantly available in the host organisms' cells. Similarly such codons may reflect transcript RNA structures that for their spatial arrangement can be more efficiently translated.

Identifying codon usage frequencies not on the basis of the whole organism, but for abundant proteins only thus opens the intriguing possibility of defining codons that will likely have a tendency to drive strong expression of a polypeptide. If such frequent codons are specifically selected and replaced by a less frequently used codon as described above, this should additionally help to reduce expression of the respective polypeptide. Moreover, if a codon is used only very rarely in abundant proteins, this may indicate that such a codon is not translated efficiently in the host organism. This presumed inability can be used for a further preferred embodiment of the present invention in which codons are purposively chosen for the modified nucleotide sequence that are only rarely used in abundant proteins of the host cell.

It seems reasonable to assume that the finding that highly expressed proteins in a host cell have a different codon usage compared to the situation where codon usage for all genes of an organism is determined will not be limited to C. glutamicum but also be observed for other organisms such as E. coli. yeast cells, plant cells, insect cells or mammalian cell culture cells.

Thus, the invention is concerned with reducing expression of polypeptides in a host cell by using modified nucleotide sequences instead of the endogenous sequences wherein, codons of the endogenous sequences are exchanged for less frequently used codons of the host organism, lids reference codon usage will preferably be determined. In a preferred embodiment codons are exchanged for the least frequently used codons of the host organism. In a further preferred embodiment, one will particularly replace those codons of the endogenous sequences which use codons that are known to be found frequently in the abundant proteins of the host organism.

The term “host cell” or “organism” for the purposes of the present invention refers to any organism that is commonly used for expression of nucleotide sequences for production of e.g. polypeptides or fine chemicals. In particular the term “host cell” or “organism” relates to prokaryotes, lower eukaryotes, plants, insect cells or mammalian cell culture systems.

The organisms of the present invention thus comprise yeasts such as S. pombe or S. cerevisiae and Pichia pastoris.

Plants are also considered by the present invention as host organisms. Such plants may be monocots or dicots such as monocotyledonous or dicotyledonous crop plants, food plants or forage plants. Examples for monocotyledonous plants are plants belonging to the genera of avena (oats), triticum (wheat), secale (rye), hordeum (barley), oryza (rice), panicum, pennisetum, setaria, sorghum (millet), zea (maize) and the like.

Dicotyledonous crop plants comprise inter alias cotton, leguminoses like pulse and in particular alfalfa, soybean, rapeseed, tomato, sugar beet, potato, ornamental plants as well as trees. Further crop plants can comprise fruits (in particular apples, pears, cherries, grapes, citrus, pineapple and bananas), oil palms, tea bushes, cacao trees and coffee trees, tobacco, sisal as well as, concerning medicinal plants, rauwolfia and digitalis. Particularly preferred are the grains wheat, rye, oats, barley, rice, maize and millet, sugar beet, rapeseed, soy, tomato, potato and tobacco. Further crop plants can be taken from U.S. Pat. No. 6,137,030.

Mammalian cell culture systems may be selected from the group comprising e.g. NIH T3cells, CHO cells, COS cells, 293 cells, Jurkat cells and HeLa cells.

Particularly preferred are microorganisms being selected from the genus of Corynebacterium with a particular locus on Corynebacterium glutamicum, the genus of Escherichia with a particular focus on Escherichia coli, the genus of Bacillus, particularly Bacillus subtilis, and the genus of Streptomyces.

As set out above, a preferred embodiment of the invention relates to the use of host cells which are selected from coryneform bacteria such as bacteria of the genus Corynebacterium. Particularly preferred are the species Corynebacterium glutamicum, Corynebacterium acetoglutamicum, Corynebacterium acetoacidophilum, Corynebacterium callunae, Corynebacterium ammoniagenes, Corynebacterium thermoaminogenes, Corynebacterium melassecola and Corynebacterium effiziens. Other preferred embodiments of the invention relate to the use of Brevibacteria and particularly the species Brevibacterium flavum, Brevibacterium lactofermentum and Brevibacterium divarecatum.

In preferred embodiments of the invention the host cells may be selected from the group comprising Corynebacterium glutamicum ATCC13032, C. acetoglutamicum ATCC15806, C. acetoacidophilum ATCC13870, Corynebacterium thermoaminogenes FERMBP-1539, Corynebacterium melassecola ATCC17965, Corynebacterium effiziens DSM 44547, Corynebacterium effiziens DSM 44549, Brevibacterium flavum ATCC14067, Brevibacterium lactoformentum ATCC13869, Brevibacterium divarecatum ATCC 14020, Corynebacterium glutamicum KFCC10065 and Corynebacterium glutamicum ATCC21608 as well as strains that are derived thereof by e.g. classical mutagenesis and selection or by directed mutagenesis.

Other particularly preferred strains of C. glutamicum may be selected from the group comprising ATCC13058, ATCC13059, ATCC13060, ATCC21492, ATCC21513, ATCC21526, ATCC21543, ATCC13287, ATCC21851, ATCC21253, ATCC21514, ATCC21516, ATCC21299, ATCC21300, ATCC39684, ATCC21488, ATCC21649, ATCC21650, ATCC19223, ATCC13869, ATCC21157, ATCC21158, ATCC21159, ATCC21355, ATCC31808, ATCC21674, ATCC21562, ATCC21563, ATCC21564, ATCC21565, ATCC21566, ATCC21567, ATCC21568, ATCC21569, ATCC21570, ATCC21571, ATCC21572, ATCC21573, ATCC21579, ATCC19049, ATCC19050, ATCC19051, ATCC19052, ATCC19053, ATCC19054, ATCC19055, ATCC19056, ATCC19057, ATCC19058, ATCC19059, ATCC19060, ATCC19185, ATCC13286, ATCC21515, ATCC21527, ATCC21544, ATCC21492, NRRL B8183, NRRL W8182, B12NRRLB12416, NRRLB12417, NRRLB12418 and NRRLB11476.

The abbreviation KFCC stands for Korean Federation of Culture Collection, ATCC stands for American-Type Strain Culture Collection and the abbreviation DSM stands for Deutsche Sammlung von Mikroorganismen. The abbreviation NRRL stands for ARS cultures collection Northern Regional Research Laboratory, Peorea, Ill., USA.

Particularly preferred are microorganisms of Corynebacterium glutamicum that are already capable of producing fine chemicals such as L-lysine, L-methionine and/or L-threonine. Therefore the strain Corynebacterium glutamicum ATCC13032 and derivatives of this strain are particularly preferred.

The term “reducing the amount of at least, one polypeptide in a host cell” refers to the situation that if one replaces an endogenous nucleotide sequence coding for a polypeptide with a modified nucleotide sequence in accordance with the invention that encodes for a polypeptide of substantially the same amino acid sequence and/or function, a reduced amount of the encoded polypeptide will be expressed within host cells. This, of course, assumes that the comparison is made for comparable host cell types, comparable genetic background situations etc.

The term “nucleotide sequence” for the purposes of the present invention relates to any nucleic acid molecule that encodes for polypeptides such as peptides, proteins etc. These nucleic acid molecules may be made of DNA, RNA or analogues thereof. However, nucleic acid molecules being made of DNA are preferred.

The terms “non-modified nucleotide sequence” or “starting nucleotide sequence” for the purposes of the present invention will typically relate to an endogenous nucleotide sequence encoding for a polypeptide the expression of which is intended to be reduced. These non-modified or starting nucleotide sequences have not been amended with respect to their codon usage, i.e. no codons have been replaced by less frequently used codons.

The terms “non-modified nucleotide sequence” and “starting nucleotide sequence” do not necessarily have to be equivalent to an endogenous nucleotide sequence. One may for example envisage the situation that an endogenous gene encoding for factor X has been deleted and replaced by a mutated version of factor X carrying a point mutation which leads to a reduced activity of this factor. However, aside from the point mutation the coding sequence of factor X may not have been amended. Starting from such a nucleotide sequence one can still further reduce expression of the mutated factor X by replacing codons of the coding sequence with less frequently used codons. Therefore, the starting nucleotide sequence will not be identical to an endogenous sequence. Nevertheless, the starting nucleotide sequence is characterized in that it has not been amended on the basis of codon usage information to lead to a reduced expression.

Thus, the terms “non-modified” or “starting nucleotide sequence” relate to a nucleotide sequence encoding for an endogenous protein or mutated versions the codon usage of which has not been amended for reducing expression of the encoded polypeptide by replacing codons with less frequently codons as determined for the respective host organism and preferably for the group of abundant proteins thereof.

The term “modified nucleotide sequence” for die purposes of the present invention relates to a sequence that has been modified with the intention to reduce expression of the encoded respective polypeptide in a host cell by adjusting the sequence of the originally different non-modified/starting nucleotide sequence. Thus, codons of the non-modified/starting sequence are replaced by less frequently used codons. The reference codon usage is based on the codon usage of the host organism.

The person skilled in the art is clearly aware that modification of the starting nucleotide sequence describes the process of optimization with respect to codon usage.

If, for example, the coding sequence of an endogenous enzyme is adjusted in order reduce expression of this factor, the changes introduced can be easily identified by comparing the modified sequence and the starting sequence which in such a case is the wild type sequence. Moreover, both sequences will encode in this case for the same amino acid sequence.

If, however, the coding sequence of e.g. an endogenous wild type enzyme is adjusted as described and if the resulting sequence is simultaneously or subsequently further amended by e.g. deleting amino acids, inserting additional amino acids or introducing point mutations in order to convey e.g. new properties to the enzyme (such as reduced feed back inhibition), the resulting modified nucleotide sequence and modified nucleotide sequence may not encode for identical amino acid sequences. In such a situation, no starting sequence in the sense that the starting sequence and the modified sequence encode for the same amino acid sequence may be present simply because the mutation which has been introduced had not been described before. Moreover, the function of the encoded may or may not be affected by the introduced mutations. If for example a point mutation is inserted in a feed back regulated enzyme, the enzyme may still catalyze the respective reaction, but it may be resistant to feed back inhibition. If e.g. amino acids are introduced at the N- or C-terminus, this may have no impact at all on the function. A skilled person will realize that despite the differences between the modified and non-modified nucleotide sequences the inventive method has been used because the starting sequence without the introduced mutation will be known in the form of wild type sequence and the differences of die modified and the starting sequence for those codons which do not code for the introduced mutation will clearly indicate that codon usage optimisation as described above has been carried out. Thus, codon usage optimisation will be clear from a comparison of the starting and the modified sequence for those codons which code for the amino acids at the same or equivalent positions.

This is meant when it is stated in the context of the present invention that the modified and starting nucleotide sequences encode for proteins of substantially identical amino acid sequence and/or function. The modified and starting nucleotide sequence will typically be at least 60%, 65%, preferably at least 70%, 75%, 80%, 85% and more preferably at least 90%, 95 or at least 98% identical as regards the amino acid sequence.

It has been set out above that in a preferred embodiment, one will not only replace codons with less frequently used codons according to the codon usage of the host organism, hut also select the codons to be replaced according to certain criteria. The replacement of codons which are frequently used in the host organism may have an additional positive effect because the increased usage frequency indicates the selection of such codons by an organism if expression is to be increased. The replacement of such frequent codons by less frequently used codons and preferably by the least frequently codons for reducing expression thus not only establishes “brakes” in the sequence but also removes elements which usually drive expression. The replacement of codons that are frequently used in abundant or ubiquitous proteins is particularly preferred.

The term “abundant proteins” for the purposes of the present invention relates to the group of highly expressed proteins within a host cell or organism.

The person skilled in the art is familiar with identifying the group of abundant proteins in a host cell or organism. This may be achieved e.g. by 2D gel electrophoresis. In 2D gel electrophoresis, a protein mixture such as a crude cellular extract is separated on protein gels by e.g. size, and isoelectric point. Subsequently these gels are stained and the intensity of the various spots is an indication of the overall amount of protein present in the cell.

Using standard software packages one will select a group of proteins whose signal intensities are above a certain threshold background level and will define this group of protein as abundant proteins. Typical software packages used for this purpose include e.g. Melanie3(Geneva Bioinformatics SA).

The person skilled in the art is well aware that different host cells such as microorganisms, plant cells, insect cells etc. will differ with respect to the number and kind of abundant proteins in a cell. Even within the same organism, different strains may show a somewhat heterogeneous expression profile on the protein level. One will therefore typically analyse different strains and consider such proteins that are found for all strains to be abundant.

A good selection parameter for defining a group of abundant proteins for the purposes of the present invention is to consider only the 10 to 300 and preferably 10 to 30 most abundant proteins as detected in the above described 2D gel electrophoresis procedure. Preferably one will only consider cytosolic proteins for the group of abundant proteins, only.

Thus, in a preferred embodiment the term “abundant proteins” refers to the group of the approximately 13, 14 or 15 abundant proteins in whole cell cytosolic extracts of host organisms as identified by 21) gel electrophoresis.

Once one has identified the abundant proteins, one may use software tools such as the “Cusp” function of the EMBOSS toolbox version 2.2.0 that can be downloaded at HTTP://EMBOSS.sourceforge.net/download/. Other software packages that, may be used are available at www.entelechon.com (e.g. Leto I.O).

As has been explained above, the principle of the invention is to reduce protein expression in a host by replacing codons of the sequence to be expressed with less frequently used codons as being apparent from the codon usage of the host organism and preferably of the abundant proteins of the host organism. In a preferred embodiment of the invention one preferentially replaces codons which are frequently found in proteins of the host organism. A particularly preferred embodiment of the invention relates to the replacement of codons as they are frequently found in abundant proteins of the host organism.

In yet another preferred embodiment, codons are replaced by rare codons or by the least frequently codons. Combinations of the aforementioned embodiments also form part of the invention.

Unless otherwise indicated the term “frequent codons” refers to the relative frequency by which a certain codon of all possible codons encoding a specific amino acid is used by the proteins of the host cell and preferably by the abundant proteins of the host cell.

A codon will be considered to be “frequent” if it is used at a relative frequency of more than 40%. It is “very frequent” if it is used at relative frequency of more than 60% and a relative frequency of more than 80% is indicative of an “extremely frequent” codon. Again the relative frequencies are based on the codon usage of proteins of the host cell unless otherwise indicated.

Unless otherwise indicated the term “rare codons” refers to the relative frequency by which a certain codon of ah possible codons encoding a specific amino acid is used by the proteins of the host cell.

A codon will he considered to be “rare” if it is used less than 20% for the specific amino acid. A “very rare” codon will be used at a frequency of less than 10% and an “extremely rare” codon will be used at a frequency of less than 5%.

As the amino acids methionine and tryptophane are encoded by one codon only, the respective codon frequency is always 100%. However the amino acid threonine is encoded by four codons, namely ACU, ACC, ACA and ACG. For the whole organism of C. glutamicum these codons are used at a relative frequency of 20.4%, 52.9% , 12.5% and 14.3% (see table 1, experiment 1). In view of the above explanations the codons ACC is thus a frequent codon and the codons ACA and ACG are rare codons.

The amino acid alanine is encoded by four codons, namely GCU, GCC, GCA and GCG. For the whole organism of C. glutamicum these codons are used at a relative frequency of 23.7%, 25.4% , 29.3% and 21.6% (see table 1, experiment 1). However, in the group of abundant proteins, these codons are used at relative frequencies of 46.8%, 9,9%, 35.9% and 7.4% (see table 2, experiment 1) Thus GCU for the group of abundant proteins turns out to be a frequent codon. On the other side GCG is particularly avoided in abundant proteins (see table 2, experiment 1). Thus, one may consider to specifically replace the codon GCU in the starting sequence with GCG in the modified sequence if reduction of expression in C. glutamicum is the goal.

In some embodiments it may be sufficient and preferred to replace codons with less frequently used codons. In a preferred embodiment the modified nucleotide sequence may use for each of the most frequently used codon the least frequently used codon.

The terms “express”, “expressing,” “expressed” and “expression” refer to expression of a gene product (e.g., a biosynthetic enzyme of a gene of a pathway) in a host organism. The expression can be done by genetic alteration of the microorganism that is used as a starting organism. In some embodiments, a microorganism can be genetically altered (e.g., genetically engineered) to express a gene product at an increased level relative to that produced by the starting microorganism or in a comparable microorganism which has not been altered. Genetic alteration includes, but is not limited to, altering or modifying regulatory sequences or sites associated with expression of a particular gene (e.g. by adding strong promoters, inducible promoters or multiple promoters or by removing regulatory sequences such that expression is constitutive), modifying the chromosomal location of a particular gene, altering nucleic acid sequences adjacent to a particular gene such as a ribosome binding site or transcription terminator, increasing the copy number of a particular gene, modifying proteins (e.g., regulatory proteins, suppressors, enhancers, transcriptional activators and the like) involved in transcription of a particular gene and/or translation of a particular gene product, or any other conventional means of deregulating expression of a particular gene using routine in the art (including but not limited to use of antisense nucleic acid molecules, for example, to block expression of repressor proteins).

The method of reducing the amount of a polypeptide in accordance with the invention comprises the step of expressing a modified nucleotide sequence which encodes for a polypeptide in the host cell instead of a starting nucleotide sequence which encodes for a polypeptide of substantially the same amino acid sequence and/or function. The modified nucleotide sequence is derived from the starting nucleotide sequence such that codons of the non-modified nucleotide sequence are exchanged in the modified nucleotide sequence with less frequently used and preferably the least frequently used codons. In a preferred embodiment frequent and preferably very or extremely frequent codons are thus exchanged for rare and preferably for very rare and extremely rare codons. In a further preferred embodiment the most frequently used codons are replaced by the least frequently used codons. In all these case is the reference codon usage based in the codon usage of the host organism and preferably on the codon usage of abundant proteins of the host organism.

In a preferred embodiment one will use a modified nucleotide sequence which uses only rare and preferably very rare and extremely rare codons for each replaced amino acid as determined for the proteins of the host cell. In a further preferred embodiment of this latter aspect of the invention the modified nucleotide sequence uses for each amino acid the least frequently used codon.

In all these cases, the start codon may be a preferred “hot spot” for introducing less frequently used codons, namely GTG instead of ATG and even more preferably TTG.

The polypeptides that are to be expressed by this method of reducing the amount of a polypeptide in a host cell can be endogenous polypeptides with the proviso that the modified nucleotide sequence must not be identical with non-modified nucleotide wild type sequences of the respective endogenous polypeptides of the host cell.

The host cells may be selected from microorganisms, insect cells, plant cells or mammalian cell culture systems. One may use the same host cell types as specified above.

Using these methods and the afore-mentioned organisms, expression of the respective polypeptide may be reduced in the host cell by at least 5%, 10%, 20%, 30%, 40%, 50%, 60%, 70%, 80%, 90% or 95%. The extent of reduction of expression is determined in comparison to the level of expression of the starting nucleotide sequence under comparable conditions.

It is understood that it is not always desirable to reduce expression as much as possible. In certain cases a repression of e.g. 25% may be sufficient and desirable. The present invention offers the possibility to fine tune repression by e.g. not replacing all codons by the least frequently used codons, but by e.g. introducing only two or three rare codons at selected positions.

The present invention also relates to modified nucleotide sequences that can be used to reduce expression of a polypeptide in a host cell.

Such modified nucleotide sequences are derived from the starting nucleotide sequences encoding for a polypeptide of substantially the same amino acid sequence and/or function with the codon usage of the modified nucleotide sequence being adjusted such that, codons of the non-modified, (wild-type) sequences are replaced by less frequently used codons. The reference codon usage will be based on the codon usage of the host organism and preferably on the codon usage of abundant proteins of the host organism.

Of course, the invention in a preferred embodiment relates to such modified nucleotide sequences that have been derived for a specific polypeptide by replacing at least one, at least two, at least three, at least four, at least five, at least six, at least seven, at least eight, at least nine, at least ten, preferably at least 1%, at least. 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequences are replaced, in the modified nucleotide sequence by less frequently used codons for the respective amino acid. In an even more preferred embodiment the afore-mentioned number of codons to be replaced refers to frequent, very frequent, extremely frequent or the most frequent codons which are replaced by rare, very rare or extremely rare conditions. In another particularly preferred embodiment, the above numbers of codons are replaced by the least frequently used codons. In all cases will the reference codon usage be based on the codon usage of the host organism and preferably on the codon usage of abundant proteins of the host organism.

In one preferred embodiment the modified nucleotide sequences will use for each replaced amino acid the least frequently used codon as determined for the proteins of the host cell.

The present invention also relates to vectors comprising such modified nucleotide sequences. These vectors can be used to express such sequences if for example the endogenous sequences have been deleted on the chromosomal level. The vectors can also be used to replace the endogenous sequences with the modified nucleotide sequences.

The present invention also relates to host cells comprising the aforementioned modified nucleotide sequences and/or the aforementioned vectors.

Particularly preferred microorganisms for performing the above method of reducing expression are selected from the genus of Corynebacterium with a particular focus on Corynebacterium glutamicum, the genus of Escherichia with a particular focus on Escherichia coli, the genus of Bacillus, particularly Bacillus subtilis, the genus of Streptomyces and the genus of Aspergillus.

A preferred embodiment of the invention relates to the use of host cells which are selected from Coryneform bacteria such as bacteria of the genus Corynebacterium. Particularly preferred are the species Corynebacterium glutamicum, Corynebacterium acetoglutamicum, Corynebacterium acetoacidophilum, Corynebacterium thermoaminogenes, Corynebacterium callunae, Corynebacterium ammoniagenes, Corynebacterium melassecola and Corynebacterium effiziens. Other preferred embodiments of the invention relate to the use of Brevibacteria and particularly the species Brevibacterium flavum, Brevibacterium lactofermentum and Brevibacterium divarecatum.

Particularly preferred are host cells selected from the group comprising Corynebacterium glutamicum ATCC13032, C. acetoglutamicum ATCC15806, C. acetoacidophilum ATCC13870, Corynebacterium thermoaminogenes FERMBP-1539, Corynebacterium melassecola ATCC17965, Corynebacterium effiziens DSM 44547, Corynebacterium effiziens DSM 44549, Brevibacterium flavum ATCC14067, Brevibacterium lactoformentum ATCC13869, Brevibacterium divarecatum ATCC 14020, Corynebacterium glutamicum KFCC10065 and Corynebacterium glutamicum ATCC21608 as well as strains that are derived thereof by e.g. classical mutagenesis and selection or by directed mutagenesis.

Moreover the invention concerns the use of the aforementioned methods, modified nucleotide sequences, vectors and/or host cells to produce fine chemicals. In a preferred embodiment to this aspect of the invention one will use modified nucleotide sequences which are selected from the group comprising nucleotide sequences encoding genes of biosynthetic pathways of fine chemicals for which repression is known to enhance production of the fine chemicals.

The term “fine chemical” is well known to the person skilled in the art and comprises compounds which can be used in different parts of the pharmaceutical industry, agricultural industry as well as in the cosmetics, food and feed industry. Fine chemicals can be the final products or intermediates which are needed for further synthesis steps. Fine chemicals also include monomers for polymer synthesis.

Fine chemicals are defined as ail molecules which contain at least two carbon atoms and additionally at least one heteroatom which is not a carbon or hydrogen atom. Preferably fine chemicals relate to molecules that comprise at least two carbon atoms and additionally at least one functional group, such as hydroxy-, amino-, thiol-, carbonyl-, carboxy-, methoxy-, ether-, ester-, amido-, phosphoester-, thioether- or thioester-group.

Fine chemicals thus preferably comprise organic acids such as lactic acid, succinic acid, tartaric acid, itaconic acid etc. Fine chemicals further comprise amino acids, purine and pyrimidine bases, nucleotides, lipids, saturated and unsaturated fatty acids such as arachidonic acid, alcohols, e.g. diols such as propandiol and butandiol, carbohydrates such as hyaluronic acid and trehalose, aromatic compounds such as vanillin, vitamins and cofactors etc.

A particularly preferred group of fine chemicals for the purposes of the present invention are biosynthetic products being selected from the group comprising organic acids, proteins, amino acids, lipids etc. Other particularly preferred fine chemicals are selected from the group of sulphur containing compounds such as thionine, cysteine, homocysteine, cystathionine, glutathione, biotine, thiamine and/or lipoic acid.

The group of most preferred fine chemical products include amino acids among which glycine, lysine, methionine, cysteine and threonine are particularly preferred.

A preferred method in accordance with the present invention relates to a method of reducing the amount of at least one polypeptide in Corynebacteria and preferably in C. glutamicum wherein the above principles are used. Thus one will express a modified nucleotide sequence instead of the starting nucleotide sequence both of which encode for substantially the same amino acid sequence and/or function wherein at least one codon of the starting nucleotide sequence is replaced in the modified nucleotide sequence by a less frequently used codon. The reference codon usage is determined for Corynebacteria and preferably for C. glutamicum. Preferably the reference codon usage is determined for the abundant proteins Corynebacteria and preferably for C. glutamicum.

Of course, the definitions as provided above for the meaning of the terms “modified nucleotide sequences”, “non-modified nucleotide sequences”, “rare codons”, “frequent codons” etc. apply equally for these preferred embodiments of the invention.

A particularly preferred embodiment of the invention relates to a method of decreasing the expression of isocitrate dehydrogenase in a microorganism by adapting the codon usage as described herein. The microorganism can be a Corynebacterium, with C. glutamicum being preferred. These methods may be used improve synthesis of amino acids and particularly of methionine and/or lysine.

The invention also relates to a host cell, which may be C. glutamicum, comprising a modified nucleotide sequence that will lead to a reduced expression of isocitrate dehydrogenase

As mentioned above, it can be preferred to replace codons which are frequently found in the group of abundant proteins. The abundant proteins of e.g. C. glutamicum can be determined as described above by 2D protein gel electrophoresis. To this purpose, C. glutamicum strains may be cultivated under standard conditions. Then, cell extracts may be prepared using common lysis protocols. After lysis, the cell extracts are centrifuged and approximately 25-50 μg are analyzed by standard 2D-PAGE. An example of the approach can be found below in example 1 as well as in the material and methods part of Hansmeier et al. (Proteomics 2006, 6, 233-250)

Following this approach abundant proteins in C. glutamicum can be identified by either selecting the most abundant 10 to 300 cytosolic proteins or by identifying 10 to 30 cytosolic proteins that are observed to be present in elevated amounts in various strains. These results are assumed to be representative also for the group of abundant proteins in other Corynebacterium species.

For the purposes of the present invention, the term “abundant proteins of C. glutamicum” can relate to the group comprising the following protein factors (accession number of nucleotide sequence shown in brackets):
Elongation Factor Tu (Genbank accession no: X7/034)
Glycerin-aldehyde-3-phosphate-dehydrogonase (Genbank accession no: BX927152, ±, nt, 289401-288397)
Fructose bisphosphate aldolase (Genbank accession no: BX927156, ±, nt. 134992-133958)
Elongation Factor Ts (Genbank accession no: BX927154, ±, nt. 14902-14075)
Hypothetical protein (Genbank accession no: BX927155, ±, nt. 213489-214325)
Enolase (Genbank accession no: BX927150, nt. 338561-339838)
Peptidyl-prolyl-Cis-trans isomerase (Genbank accession no: BX927148, nt, 34330-34902)
Superoxide dismutase (Genbank accession no: AB055218)
Phosphoglycerate dehydrogenase (Genbank accession no: BX92715L nt. 306039-307631)
SSU Rib protein SIP (Genbank accession no: BX927152, ±, nt. 26874-28334)
Triose phosphate-isomerase (Genbank accession no: BX927152, ±, nt. 286884-286105)
Isopropylmalat-synthase (Genbank accession no: X70959)
Butane-2,3-dioldehydrogenase (Genbank accession no: BX927156, nt. 20798-21574)
Fumarate-hydratase (Genbank accession no: BX927151, ±, nt. 18803-17394)

On the basis of these aforementioned fourteen proteins, a codon usage table can be created using the aforementioned “CUSP” function of the EMBOSS toolbox

The above described group of fourteen proteins may particularly be used for determining or for defining the group of abundant proteins in C. glutamicum if the C. glutamicum strain ATCC 13032 and/or derivatives (obtained e.g. by classical mutagenesis and selection or genetic engineering) are used in the 2D-gel electrophoresis analysis.

Using the CUSP function of the EMBOSS toolbox version one can thus create a Codon Usage Table that reflects codon usage of abundant proteins of Corynebacterium in general and preferably of C. glutamicum.

Surprisingly the codon usage of these abundant proteins differs significantly from the codon usage as determined for the whole genome of C. glutamicum as becomes clear from a comparison of tables 1 and 2 (see Experiment 1 below). Codon usage of the whole genome of C. glutamicum can e.g. be determined from strains that are completely sequenced such as strain ATCC13032 and Codon Usage Tables may e.g. generated by the CUSP function of the aforementioned EMBOSS toolbox or are available at e.g. HTTP://www.kazusa.or.jp. Highly comparable results are obtained if one uses the most abundant cytosolic proteins as mentioned in Table 4 of Hansmeier et al, (vide supra).

A preferred embodiment of the aspect of the invention that, relates to methods of reducing the amount of polypeptides in a host cell derived from the genus Corynebacterium and preferably from the species Corynebacterium glutamicum comprises the step of expressing in Corynebacterium and preferably in C. glutamicum a modified nucleotide sequence encoding for at least one polypeptide instead of an endogenous non-modified nucleotide sequence encoding for a polypeptide of substantially the same amino acid sequence and/or function wherein said modified nucleotide sequence is derived from the starting nucleotide sequence such that one, some or preferably all of the codons of the starting nucleotide sequences are replaced in the modified nucleotide sequence by less frequently used codons for the respective amino acid. In an even more preferred embodiment the codons to be replaced are frequent, very frequent or extremely frequent codons. In a particularly preferred embodiment these codons are replaced by rare, very rare or extremely rare codons. In another particularly preferred embodiment, the above numbers of codons are replaced by the least frequently used codons, respectively. In all these cases will the reference codon usage be based on the codon usage of Corynebacterium and preferably on C. glutamicum. Preferably, the reference codon usage is determined for the abundant proteins of Corynebacteria and preferably of C. glutamicum.

For reducing expression of polypeptides in the genus of Corynebacterium and particularly in the species of C. glutamicum by optimised codon usage, in another embodiment the non-modified nucleotide sequence encoding for the polypeptide may be modified such that at least one, at least two, at least three, at least four, at least five, at least six, at least seven, at least eight, at least nine, at least ten, preferably at least 1%, at least 2%, at least 4%, at least 6%. at least 8%, at least 10%, more preferably at least 20%, at least 40%, at least 60%, at least 80%, even more preferably at least 90% or least 95% and most preferably all of the codons of the non-modified nucleotide sequence are replaced in the resulting modified nucleotide sequence by less frequently used codons for the respective amino acid according to Table 1 and preferably according to Table 2.

In yet another embodiment of the invention wherein the method is used to reduce the amount of polypeptides in the genus Corynebacterium and preferably in C. glutamicum the codons of the modified nucleotide sequence are selected from one of the two least frequently used codons encoding for the respective amino acid(s) as set forth in Table 1 and preferably in Table 2.

In yet another embodiment of the invention which relates to a method of decreasing the amount of polypeptides in Corynebacterium and particularly preferably in C. glutamicum, at least one, some or all codons of the modified nucleotide sequences may be selected from the codon usage of Table 3.

In another embodiment of the invention, the method may rely on modified nucleotide sequences which use the codons ACG for threonine, GAT for aspartic acid, GAA for glutamic acid, AGA and AGG for arginine and/or TTG for the start codon.

Another particularly preferred embodiment of the invention relates to methods of decreasing the amount of a polypeptide in Corynebacterium and particularly preferred in C. glutamicum wherein at least one, at least two, at least three, at least four, at least live, at least six, at least seven, at least eight, at least nine, at least ten, preferably at least 1%, at least 2%, at least 4%, at least 6%, at least 8%, at least 10%, more preferably at least 20%, at least 40%, at least 50%, at least 80%, even more preferably at least 90% or least 95% and most preferably all of the codons of the non-modified nucleotide sequence are replaced in the resulting modified nucleotide sequence by the codons for the respective amino acid according to Table 3 and preferably according to Table 4. In an even more preferred embodiment the afore-mentioned number of codons to be replaced refers to frequent, very frequent, extremely frequent or the most frequent codons.

Another particularly preferred embodiment of the invention relate to methods of decreasing the amount of a polypeptide in Corynebacterium and particularly preferred in C. glutamicum wherein ail codons of the non-modified nucleotide sequence are replaced in the resulting modified nucleotide sequence by the codons for the respective amino acid according to Table 3 and preferably according to Table 4.

These methods may particularly used for reducing expression in host cells selected from the Corynebacteria mentioned above. The start codon may be a preferred “hot spot” for introducing less frequently used codons, namely GTG instead of ATG and even more preferably TTG.

The host organism may be selected also from the group comprising Corynebacterium glutamicum, Corynebacterium acetoglutamicum, Corynebacterium acetoacidophilum, Corynebacterium thermoaminogenes, Corynebacterium melassecola and Corynebacterium effiziens.

Also preferred are the above-mentioned C. glutamicum strain and particularly preferred is the strain Corynebacterium glutamicum ATCC13032 and all its derivatives. The strains ATCC 13286, ATCC 13287, ATCC 21086. ATCC 21127, ATCC 21128, ATCC 21129, ATCC 21253, ATCC 21299, ATCC 21300, ATCC 21474, ATCC 21475, ATCC 21488, ATCC 21492, ATCC 21513, ATCC 21514, ATCC 21515, ATCC 21516, ATCC 21517, ATCC 21518, ATCC 21528, ATCC 21543, ATCC 21544, ATCC 21649, ATCC 21650, ATCC 21792, ATCC 21793, ATCC 21798, ATCC 21799, ATCC 21800, ATCC 21801, ATCC 700239, ATCC 21529, ATCC 21527, ATCC 31269 and ATCC 21526 which are known to produce lysine can also preferably be used. The other aforementioned strains can also be used.

The extent of reduction of protein expression may be the same as mentioned above.

One aspect of the invention also relates to (modified) nucleotide sequences as they may be used in the above-described methods of reducing the amount of a polypeptide in Corynebacterium and preferably in C. glutamicum.

In case of modified nucleotide sequences that are to be expressed in Corynebacterium and particularly preferably in C. glutamicum for reducing the amount of the respective encoded polypeptide, at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequences may be replaced in the modified nucleotide sequence by less frequently used codons for the respective amino acid. In an even more preferred embodiment the afore-mentioned number of codons to be replaced refers to frequent, very frequent, extremely frequent or the most frequent codons which are replaced by rare, very rare or extremely rare codons. In another particularly preferred embodiment, the above number of codons replaced by the least frequently used codons. In all these cases will the reference codon usage be based on the codon usage of Corynebacterium and preferably on C. glutamicum. Preferably, the reference codon usage is based on the codon usage of abundant proteins of Corynebacterium and preferably of C. glutamicum.

In case of modified nucleotide sequences that are to be expressed in Corynebacterium and particularly preferably in C. glutamicum for reducing the amount of the respective encoded polypeptide, at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%), even more preferably at least 90% or least 95% and most preferably ail of the codons of the non-modified nucleotide sequence are replaced in the resulting modified nucleotide sequence by less frequently used codons for the respective amino acid according to Table 1 and preferably according to Table 2. In an even more preferred embodiment the afore-mentioned number of codons to be replaced refers to frequent, very frequent, extremely frequent or the most frequent codons. In another particularly preferred embodiment, the afore-mentioned numbers of codons are replaced by the least frequently used codons of Table 1 and preferably of Table 2.

For other preferred embodiments as far as nucleotide sequences for reducing expression in Corynebacterium and particularly preferably in C. glutamicum is concerned, at least one, at least two, at least three, at least four, at least five, at least six, at least seven, at least eight, at least nine, at least ten, preferably at least 1%, at least 2%, at least 4%, at least 6%, at least 8%, at least 10%, more preferably at least 20%, at least 40%, at least 60%, at least 80%, even more preferably at least 90% or least 95% and most preferably all of the codons of the non-modified nucleotide sequence are replaced in the resulting modified nucleotide sequence by one of the two least frequently used codons for the respective amino acid according to Table 1and preferably according to liable 2. In an even more preferred embodiment the afore-mentioned number of codons to be replaced refers to frequent, very frequent, extremely frequent or the most frequent codons. The estimation of whether a codon is frequent, very frequent or extremely frequent will preferably be based on the codons usage of abundant proteins of Corynebacterium and preferably of C. glutamicum as depicted in Table 2.

In another embodiment of the invention, the modified nucleotide sequences uses the codons ACG for threonine, GAT for aspartic acid, GAA for glutamic acid, AGA and AGG for arginine and/or TTG for the start codon.

In yet another embodiment of the invention the modified nucleotide sequence which is used for reducing expression of a polypeptide in Corynebacterium and particularly preferably in C. glutamicum may comprise at least one, some or all codons being selected from Table 3 and preferably from Table 4.

Thus, another particularly preferred embodiment of the invention relates to modified nucleotide sequences for reducing expression of a polypeptide in Corynebacterium and particularly preferred in C. glutamicum wherein at least one, at least two, at least three, at least four, at least five, at least six, at least seven, at least eight, at least nine, at least ten preferably at least 1%, at least 2%, at least 4%, at least 6%, at least 8%, at least 10%, more preferably at least 20%, at least 40%, at least 60%, at least 80%>, even more preferably at least 90% or least 95% and most preferably all of the codons of the non-modified nucleotide sequence are replaced in the resulting modified nucleotide sequence by the codons for the respective amino acid according to Table 3 and. preferably according to Table 4. In an even more preferred embodiment the afore-mentioned number of codons to be replaced refers to frequent, very frequent, extremely frequent and most frequent codons.

Another particularly preferred embodiment of the invention relates to modified nucleotide sequences for reducing expression of a polypeptide in Corynebacterium and particularly preferably in C. glutamicum wherein all codons of the non-modified nucleotide sequence are replaced in the resulting modified nucleotide sequence by the codons for the respective amino acid according to Table 3 and preferably according to Table 4.

In all these case the start codon may be a preferred “hot spot” for replacing less frequently used codons, namely GTG instead of ATG and even more preferably TTG. This aspect particularly relates to decreasing the amount and/or activity of isocitrate dehydrogenase e.g. to improve methionine synthesis.

The present, invention also relates to vectors comprising the aforementioned modified nucleotide sequences which can be used to lower protein expression in Corynebacterium and preferably in C. glutamicum.

A vector that comprises the aforementioned nucleotide sequences is used to drive expression of a modified nucleotide sequence in the host cell, preferably in Corynebacterium and particularly preferably in C. glutamicum for increasing the amount of a polypeptide in these host cells. Such vectors may e.g. be plasmid vectors which are autonomously replicable in coryneform bacteria. Examples are pZ1 (Menkel et al. (1989), Applied and Environmental Microbiology 64: 549-554), pEKEx1 (Eikmanns et al. (1991), Gene 102: 93-98), pHS2-1 (Sonnen et al. (1991), Gene 107: 69-74) These vectors are based on the cryptic plasmids pHM1519, pBL1 oder pGA1. Other vectors are pCLiK5MCS (WO2005059093), or vectors based on pCG4 (U.S. Pat. No. 4,489,160) or pNG2 (Serwold-Davis et al. (1990), FEMS Microbiology Letters 66, 119-124) or pAG1 (U.S. Pat. No. 5,158,891). Examples for other suitable vectors can be found in the Handbook of Corynebacterium (edited by Eggeling and Bott, ISBN 0-84934821-1, 2005).

When optimizing the codon usage, other influencing factors, like the resulting mRNA structure, should also be considered.

The present invention also relates to host cells comprising the aforementioned vectors and modified nucleotide sequences which can be used to lower protein expression in Corynebacterium and preferably in C. glutamicum.

A person skilled in the art is familiar with how to replace e.g. a gene or endogenous nucleotide sequence that encode for a certain polypeptide with a modified nucleotide sequence. This may e.g. be achieved by introduction of a suitable construct (plasmid without origin of replication, linear DNA fragment without origin of replication) by electroporation, chemical transformation, conjugation or other suitable transformation methods. This is followed by e.g. homologous recombination using selectable markers which ensure that only such cells are identified that carry the modified nucleotide sequence instead of the endogenous naturally occurring sequence. Other methods include gene disruption of the endogenous chromosomal locus and expression of the modified sequences from e.g. plasmids. Yet other methods include e.g. transposition. Further information as to vectors and host cells that may be used will be given below.

The above described methods of reducing the amount of a polypeptide in a host cell and particularly for reducing the amount of a polypeptide in the genus Corynebacterium and even more preferably in C. glutamicum may be performed with the modified nucleotide sequence being selected from the group comprising nucleotide sequences encoding genes of biosynthetic pathways of fine chemicals if repression of expression of these genes is known to enhance fine chemical production. This is especially useful when the polypeptide is responsible for the synthesis of unwanted by-products/side products. “Lowering the amount of a polypeptide such as an enzyme that forms part of a biosynthetic pathway of the aforementioned fine chemicals may allow increasing synthesis of the fine chemicals by e.g. shutting off production of by-products and by channelling metabolic flux into a preferred direction,

Similarly the aforementioned modified nucleotide sequences, vectors and host cells can be used for producing fine chemicals in Corynebacterium and preferably in C. glutamicum.

A vector that can be used to replace an endogenous sequence with the modified sequence may also form a part of the invention. One may of course also use a vector that is suitable for expression of such nucleotide sequences in the respective host cell. This may e.g. be done if the modified sequence is expressed from a vector and the endogenous sequence has been silenced before by e.g. gene disruption.

Strains of Corynebacterium glutamicum that are already capable of producing fine chemicals such as L-lysine, L-methionine and/or L-threonine are preferred in this context. Such a strain is e.g. Corynebacterium glutamicum ATCC13032 and derivatives thereof. The strains ATCC 13286, ATCC 13287, ATCC 21086, ATCC 21127, ATCC 21128, ATCC 21129, ATCC 21253, ATCC 21299, ATCC 21300, ATCC 21474, ATCC 21475, ATCC 21488, ATCC 21492, ATCC 21513, ATCC 21514, ATCC 21515, ATCC 21516, ATCC 21517, ATCC 21518, ATCC 21528, ATCC 21543, ATCC 21544, ATCC 21649, ATCC 21650, ATCC 21792, ATCC 21793, ATCC 21798, ATCC 21799, ATCC 21800, ATCC 21801, ATCC 700239, ATCC 21529, ATCC 21527, ATCC 31269 and ATCC 21526 which are known to produce lysine can also preferably be used. The other aforementioned strains can also be used.

These modified nucleotide sequences may again be preferably selected from the group comprising nucleotide sequences encoding genes of biosynthetic pathways of fine chemicals. The definitions and preferences as to the meaning and desirability of fine chemicals given above equally apply. Thus, the group of most preferred fine chemical products includes amino acids among which glycine, lysine, methionine, cysteine and threonine are particularly preferred.

In a preferred embodiment of the methods of reducing the amount of a polypeptide in the genus Corynebacterium and particularly in C. glutamicum, the modified nucleotide sequences may thus be selected from the group comprising sequences encoding threonine-dehydratase, homoserine-O-acetyltransferase, O-acetylhomoserine-sulfhydrylase, phosphoenolpyruvate-carboxykinase (pepCK), pyruvate-oxidase (poxB), homoserine-kinase, homoserine-dehydrogenase, threonine-exporter, threonine-efflux, asparaginase, aspartate-decarboxylase and threonine-synthase, citrate synthase, aconitase, isocitrate-dehydrogenase, alpha-ketoglutarate dehydrogenase, succinyl-CoA-synthase, succinate-dehydrogenase, fumarase, malate-quinone oxidoreductase, malate dehydrogenase, pyruvate kinase, malic enzyme, air (alanine racemase), atr43 (ABC transporter), ccpA1 (catabolite control protein a), ccpA2 (catabolite control protein), chrA (two component response regulator), chrS (histidine kinase), citB (transcriptional regulator), citE (citratlyase E), citE (citrat lyase E), clpC (protease), csp1, ctaF (4. subunit of cytochrome aa3 oxidase), dctA (C4-dicarboxylat transport protein), dctQ sodit (C4-dicarboxylat transport, protein), dead (DNA/RNA helicase), def (peptide deformylase), dep33 (multi drug resistance protein B), dep34 (efflux protein), fda (fructose bisphosphate ldolase), gorA (glutathion reductase), gpi/pgi (glucose-6-P-isomerase), hisC2(histidinol phosphate aminotransferase), horn (homoserin dehydrogenase), lipA (lipoate synthase), lipB (lipoprotein-ligase B), lrp (leucine resonse regulator), luxR (transcriptional regulator), luxS (sensory transduction protein kinase), lysR1 (transcriptional regulator), lysR2(transcriptional regulator, lysR3 (transcriptional regulator), mdhA (malate dehydrogenase), menE (O-succinylbenzoic acid CoA ligase), mikE17 (transcription factor), mqo (malate-qninon oxodoreductase), mtrA mtrB (sensor protein cpxA, regulatory component of sensory), nadA (quinolinate synthase A), nadC (nicotinate nucleotide pyrophosphase), otsA (trehalose-6-P-synthase), otsB, treY, treZ (trehalose phosphatase, maltooligosyl-trehalose synthase maltooligosyl-trehalose trehalohydrolase), pepC (aminopeptidase I), pfKA pfkB (1und 6-phosphofructokinase), poxB (pyruvate oxidase), pstC2 (membrane bound phosphate transport protein), rplK (PS1-protein), sucC sucD (succinyl CoA synthetase), sugA (sugar transport protein), tmk (thymidylate kinase), zwa2, metK metZ, glyA (serinhydroxymethyltransferase), sdhC sdhA sdhR (succinat DH), smtB (transcriptional regulator), cgl1 (transcriptional regulator), hspR (transcriptional regulator), cgl2 (transcriptional regulator), cebR (transcriptional regulator), cgl3 (transcriptional regulator), gatR (transcriptional regulator), glcR (transcriptional regulator), tcmR (transcriptional regulator), smtB2 (transcriptional regulator), dtxR (transcriptional regulator), degA (transcriptional regulator), galR (transcriptional regulator), tipA2 (transcriptional regulator), mall (transcriptional regulator), cgl4 (transcriptional regulator), arsR (transcriptional regulator), merR (transcriptional regulator), hrcA (transcriptional regulator), glpR2(transcriptional regulator), lexA (transcriptional regulator), ccpA3 (transcriptional regulator), degA2 (transcriptional regulator) in the case of lysine.

In a further preferred embodiment of the methods of reducing the amount of a polypeptide in the genus Corynebacterium and particularly in C. glutamicum , the modified nucleotide sequences may thus be selected from the group comprising sequences encoding homoserine-kinase, threonine-dehydratase, threonine-synthase, meso-diaminopimelat D-dehydrogenase, phosphoenolpyruvate-carboxykinase, pyruvat-oxidase, dihydrodipicolinate-synthase, dihydrodipicolinate-reductase, and diaminopicolinate-decarboxylase in the case of methionine. Isocitrate dehydrogenase may be preferred particularly when production of methionine is concerned.

In a preferred embodiment of the methods of reducing the amount of a polypeptide in the genus Corynebacterium and particularly in C. glutamicum, the modified nucleotide sequences may thus be selected from the group comprising sequences encoding threonine-dehydratase, homoserine O-acetyltransferase, serine-hydroxymethyltransferase, O-acetylhomoserine-sulfhydrylase, meso-diaminopimelate D-dehydrogenase, phosphoenolpyruvate-carboxykinase, pyruvate-oxidase, dihydrodipicolinate-synthetase, dihydrodipicolinate-reductase, asparaginase, aspartate-decarboxylase, lysin-exporter, acetolactate-synthase, ketol-aid-reductoisomerase, branched chain aminotransferase, coenzym B12-dependent methionine-synthase, coenzym B12-independent methione-synthase, dihydroxy acid dehydratase and diaminopicolinate-decarboxylase in the case of threonine.

In general, the person skilled in the art is familiar with designing constructs such as vectors for driving expression of a polypeptide in microorganisms such as E. coli and C. glutamicum. The person skilled in the art is also well acquainted with culture conditions of microorganisms such as C. glutamicum and E. coli as well as with procedures for harvesting and purifying fine chemicals such as amino acids and particularly lysine, methionine and threonine from the aforementioned microorganisms. Some of these aspects will be set out in farther detail below.

The person skilled in the art is also well familiar with techniques that allow to change the original non-modified nucleotide sequence into a modified nucleotide sequence encoding for polypeptides of identical amino acid but with different codon usage. This may e.g. be achieved by polymerase chain reaction based mutagenesis techniques, by commonly known cloning procedures, by chemical synthesis etc. Some of these procedures are set out in the examples.

In the following, it will be described and set out in detail how genetic manipulations in microorganisms such, as E. coli and particularly Corynebacterium glutamicum can be performed.

Vectors and Host Cells

One aspect of the invention pertains to vectors, preferably expression vectors, containing a modified nucleotide sequences as mentioned above. As used herein, the term “vector” refers to a nucleic acid molecule capable of transporting another nucleic acid to which it has been linked.

One type of vector is a “plasmid”, which refers to a circular double stranded DNA loop into which additional DNA segments can be ligated. Another type of vector is a viral vector, wherein additional DNA segments can be ligated into the viral genome.

Certain vectors are capable of autonomous replication in a host cell into which they are introduced (e.g., bacterial vectors having a bacterial origin of replication and episomal mammalian vectors). Other vectors (e. g., non-episomal mammalian vectors) are integrated into the genome of a host cell upon introduction into the host cell, and thereby are replicated along with the host genome. Moreover, certain vectors are capable of directing the expression of genes to which they are operatively linked.

Such vectors are referred to herein as “expression vectors”.

In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e. g., replication defective retroviruses, adenoviruses and adeno-associated viruses), which serve equivalent functions.

The recombinant expression vectors of the invention may comprise a modified nucleic acid as mentioned above in a form suitable for expression of the respective nucleic acid in a host cell, which means that the recombinant expression vectors include one or more regulatory sequences, selected on the basis of the host cells to be used for expression, which is operatively linked to the nucleic acid sequence to he expressed.

Within a recombinant expression vector, “operably linked” is intended to mean that the nucleotide sequence of interest is linked to the regulatory sequence (s) in a manner which allows for expression of the nucleotide sequence (e.g., in an in vitro transcription/translation system or in a host cell when the vector is introduced into the host cell). The term “regulatory sequence” is intended to include promoters, repressor binding sites, activator binding sites, enhancers and other expression control elements (e.g., terminators, polyadenylation signals, or other elements of mRNA secondary structure). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in marry types of host cell and those which direct expression of the nucleotide sequence only in certain host cells. Preferred regulatory sequences are, for example, promoters such as cos-, tac-, trp-, tet-, trp-, tet-, lpp-, lac-, lpp- lac-, lacIq-, T7-, T5-, T3-, gal-, trc-, ara-, SP6-, arny, SP02, e-Pp-ore PL, SOD, EFTu, EFTs, GroEL, MetZ (last 5 from C. glutamicum), which are used preferably in bacteria. Additional regulatory sequences are, for example, promoters from yeasts and fungi, such as ADC1, MFa, AC, P-60, CYC1, GAPDH, TEF, rp28, ADH, promoters from plants such as CaMV/35S, SSU, OCS, lib4, usp, STLS1, B33, nos or ubiquitin-or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by the above-mentioned modified nucleotide sequences.

The recombinant expression vectors of the invention can be designed for expression of the modified nucleotide sequences as mentioned above in prokaryotic or eukaryotic cells. For example, the modified nucleotide sequences as mentioned above can be expressed in bacterial cells such as C. glutamicum and E. coli, insect cells (using baculovirus expression vectors), yeast and other fungal cells (see Romanes, M. A. et al. (1992), Yeast 8: 423-488; van den Hondel, C. A. M. J. J. et al. (1991) in: More Gene Manipulations in Fungi, J. W. Bennet & L. L. Lasure, eds., p. 396-428: Academic Press: San Diego: and van den Hondel, C. A. M. J. J. & Punt, P. J. (1991) in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) Plant Cell Rep.: 583-586). Suitable host cells are discussed further in Goeddel, Gene Expression Technology: Methods in Enzymology 185, Academic Press. San Diego, Calif. (1990). Alternatively, the recombinant expression vector can be transcribed and translated in vitro, for example using T7 promoter regulatory sequences and T7polymerase.

Expression of proteins in prokaryotes is most often carried out with vectors containing constitutive or inducible promoters directing the expression of either fusion or non-fusion proteins.

Fusion vectors add a number of amino acids to a protein encoded therein, usually to the amino terminus of the recombinant protein but also to the C-terminus or fused within suitable regions in the proteins. Such fusion vectors typically serve four purposes: 1) to increase expression of recombinant protein; 2) to increase the solubility of the recombinant protein; and 3) to aid in the purification of the recombinant protein by acting as a ligand in affinity purification 4) to provide a “tag” for later detection of the protein. Often, in fusion expression vectors, a proteolytic cleavage site is introduced at the junction of the fusion moiety and the recombinant protein to enable separation of the recombinant, protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein. A, respectively.

Examples of suitable inducible non-fusion E. coli expression vectors include pTrc (Amann et al., (1988) Gene 69: 301-315), pLG338, pACYC184, pBR322, pUC18, pUC19, pKC30, pRep4, pHS1, pHS2, pPLc236, pMBL24, pLG200, pUR290, pIN-III1 13-B1, egt11, pBdCl, and pET lld (Studier et al., Gene Expression Technology: Methods in Enzymology 185, Academic Press, San Diego, Calif. (1990) 60-89; and Pouwels et ah, eds. (1985) Cloning Vectors. Elsevier: New York IBSN 0 444 904018). Target gene expression from the pTrc vector relies on host RNA polymerase transcription from a hybrid trp-lac fusion promoter. Target gene expression from the pET lld vector relies on transcription from a T7 gnlO-lac fusion promoter mediated by a coexpressed viral RNA polymerase (T7gnl). This viral polymerase is supplied by host strains BL21 (DE3) or HMS174 (DE3) from a resident X prophage harboring a T7gnl gene under the transcriptional control of the lacUV 5 promoter. For transformation of other varieties of bacteria, appropriate vectors may be selected. For example, the plasmids pIJ101, pIJ364, pIJ702 and pIJ361 are known to be useful in transforming Streptomyces, while plasmids pUB110, pC194 or pBD214 are suited for transformation of Bacillus species. Several plasmids of use in the transfer of genetic information into Corynebacterium include pHM1519, pBL1, pSA77 or pAJ667 (Pouwels et ah, eds. (1985) Cloning Vectors, Elsevier: New York IBSN 0 444 904018).

Examples of suitable C. glutamicum and E. coli shuttle vectors are e.g. pClik5aMCS (WO2005059093) or can be found in Eikmanns et al (Gene (1991) 102, 93-8).

Examples for suitable vectors to manipulate Corynebacteria can be found in the Handbook of Corynebacterium (edited by Eggeling and Bott, ISBN 0-8493-1821-1, 2005). One can find a list of E. coli-C. glutamicum shuttle vectors (table 23.1), a list of E. coli-C. glutamicum shuttle expression vectors (table 23.2), a list of vectors which can be used for the integration of DNA into the C. glutamicum chromosome (table 23.3), a list of expression vectors for integration into the C. glutamicum chromosome (table 23.4.) as well as a list of vectors for site-specific integration into the C. glutamicum chromosome (table 23.6).

In another embodiment, the protein expression vector is a yeast expression vector. Examples of vectors for expression in yeast S. cerevisiae include pYepSec1 (Baldari, et al., (1987) Embo J. 6: 229-234), 2i, pAG-1, Yep6, Yep13, pEMBLYe23, pMFa (Kurjan and Herskowitz, (1982) Cell 30: 933-943), pJRY88 (Schultz et al., (1987) Gene 54: 113-123), and pYES2(Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) in: Applied Molecular Genetics of Fungi, J. F. Peberdy, et al., eds., p, 1-28, Cambridge University Press: Cambridge, and Pouwels et al., eds. (1985) Cloning Vectors. Elsevier: New York (IBSN 0 444 904018).

For the purposes of the present invention, an operative link is understood to be the sequential arrangement of promoter, coding sequence, terminator and, optionally, further regulatory elements in such a way that, each of the regulatory elements can fulfill its function, according to its determination, when expressing the coding sequence.

In another embodiment, the modified nucleotide sequences as mentioned above may be expressed in unicellular plant, cells (such as algae) or in plant cells from higher plants (e. g., the spermatophytes, such as crop plants). Examples of plant expression vectors include those detailed in: Becker, D., Kemper, E., Schell, J. and Masterson, R. (1992) Plant Mol. Biol. 20: 1195-1197; and Bevan, M. W. (1984) Nucl. Acid. Res. 12: 8711-8721, and include pLGV23, pGHlac+, pBIN19, pAK2004, and pDH51 (Pouwels et al., eds. (1985) Cloning Vectors. Elsevier: New York IBSN 0 444 904018).

For other suitable expression systems for both prokaryotic and eukaryotic cells see chapters 16 and 17 of Sambrook, J. et al. Molecular Cloning: A Laboratory Manual. 3rd ed., Cold Spring Harbor Laboratory, Cold Spring Harbor Laboratory Press, Cold Spring Harbor, N.Y., 2003.

In another embodiment, the recombinant mammalian expression vector is capable of directing expression of the nucleic acid preferentially in a particular cell type, e.g. in plant cells (e. g., tissue-specific regulatory elements are used to express the nucleic acid). Tissue-specific regulatory elements are known in the art.

Another aspect of the invention pertains to organisms or host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e. g., linear DNA or RNA (e. g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, conjugation chemical-mediated transfer, or electroporation. Suitable methods for transforming or transfecting host cells can be found in Sambrook, et al. (Molecular Cloning: A Laboratory Manual 3rd ed., (fold Spring Harbor Laboratory, Cold Spring Harbor Laboratory Press, Cold Spring Harbor, N.Y., 2003), and other laboratory manuals.

In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin, kanamycine, tetracycline, ampicillin and methotrexate. Nucleic acid encoding a selectable marker can be introduced into a host cell on the same vector as that encoding the above-mentioned modified nucleotide sequences or can be introduced on a separate vector. Cells stably transfected with the introduced nucleic acid can be identified by drug selection (e.g., cells that have incorporated the selectable marker gene will survive, while the other cells die).

When plasmids without an origin of replication and two different marker genes are used (e.g. pClik int sacB), it is also possible to generate marker-free strains which have part of the insert inserted into the genome. This is achieved by two consecutive events of homologous recombination (see also Becker et al., APPLIED AND ENVIRONMENTAL MICROBIOLOGY, 71 (12), p. 8587-8596). The sequence of plasmid pClik int sacB can be found in WO2005059093; SEQ ID 24; the plasmid is called pCIS in this document).

In another embodiment, recombinant microorganisms can be produced which contain selected, systems which allow for regulated expression of the introduced gene. For example, inclusion of one of the above-mentioned optimized nucleotide sequences on a vector placing it under control of the lac operon permits expression of the gene only in the presence of IPTG. Such regulatory systems are well known in the art.

In one embodiment, the method comprises culturing the organisms of invention (into which a recombinant expression vector or into which genome has been introduced a gene comprising the modified nucleotide sequences as mentioned above) in a suitable medium for fine chemical production. In another embodiment, the method further comprises isolating the fine chemical from the medium or the host cell.

Growth of Escherichia coli and Corynebacterium glutamicum-Media and Culture Conditions

The person skilled in the art is familiar with the cultivation of common microorganisms such as C. glutamicum and E. coli. Thus, a general teaching will he given below as to the cultivation of C. glutamicum. Corresponding information may be retrieved from standard textbooks for cultivation of E. coli.

E. coli strains are routinely grown in MB and LB broth, respectively (Follettie et al. (1993) J. Bacterial. 175, 4096-4103). Minimal media for E. coli is M9 and modified MCGC (Yoshihama et al. (1985) J. Bacteriol. 162, 591-507), respectively. Glucose may be added at a final concentration of 1%. Antibiotics may be added in the following amounts (micrograms per millilitre): ampicillin, 50; kanamycin, 25; nalidixic acid, 25. Amino acids, vitamins, and other supplements may be added in the following amounts: methionine, 9.3 mM; arginine, 9.3mM; histidine, 9.3 mM; thiamine, 0.05 mM. E. coli cells are routinely grown at 37 C, respectively.

Genetically modified Corynebacteria are typically cultured in synthetic or natural growth media. A number of different growth media for Corynebacteria are both well-known and readily available (Lieb et al. (1989) Appl. Microbiol. Bioiechnol., 32: 205-210; von der Osten et al. (1998) Biotechnology Letters, 11: 11-16; Patent DE 4,120,867; Liebl (1992) “The Genus Corynebacterium, in: The Procaryotes, Volume II, Balows, A. et al., eds. Springer-Verlag). Instructions can also be found in the Handbook of Corynebacterium (edited by Eggeling and Bott, ISBN 0-8493-1821-1, 2005).

These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribose, lactose, maltose, sucrose, glycerol, raffinose, starch or cellulose serve as very good carbon sources.

It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which, contain these compounds. Exemplary nitrogen sources include ammonia gas or ammonia salts, such as NH₄Cl or (NH₄)₂S0₄, NH₄0H, nitrates, urea, amino acids or complex nitrogen sources like corn steep liquor, soy bean flour, soy bean protein, yeast extract, meat extract and others.

The overproduction of methionine is possible using different sulfur sources. Sulfates, thiosulfates, sulfites and also more reduced sulfur sources like H₂S and sulfides and derivatives can be used. Also organic sulfur sources like methyl mercaptan, thioglycolates, thiocyanates, and thiourea, sulfur containing amino acids like cysteine and other sulfur containing compounds can be used to achieve efficient methionine production. Formate may also be possible as a supplement as are other C1 sources such as methanol or formaldehyde.

Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechtiate, or organic acids, such as citric acid. It is typical for the media to also contain other growth factors, such as vitamins or growth promoters, examples of which include biotin, riboflavin, thiamine, folic acid, nicotinic acid, pantothenate and pyridoxine. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (Eds. P. M. Rhodes, P. P. Stanbury, IRL Press (1997) PP. 53-73, ISBN 0 19 963577 3). It is also possible to select growth media from commercial suppliers, like standard 1 (Merck) or BHI (grain heart infusion, DIFCO) or others.

All medium components should be sterilized, either by heat (20 minutes at 1.5 bar and 121 C) or by sterile filtration. The components can either be sterilized together or, if necessary, separately.

All media components may be present at the beginning of growth, or they can optionally be added continuously or batch wise. Culture conditions are defined separately for each experiment.

The temperature should be in a range between 15° C. and 45° C. The temperature can be kept constant or can be altered during the experiment. The pH of the medium may be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the microorganisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microliter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD600 of 0.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 30 C.

Inoculation of the media is accomplished, by either introduction of a saline suspension of C. glutamicum cells from CM plates or addition of a liquid preculture of this bacterium.

Quantification of Amino Acids and Methionine Intermediates

The analysis is done by HPLC (Agilent 1100, Agilent, Waldbronn, Germany) with a guard cartridge and a Synergi 4 μm column (MAX-RP 80 Å, 150*4.6 mm) (Phenomenex, Aschaffenburg, Germany). Prior to injection the analytes are derivatized using o-phthaldialdehyde (OPA) and mercaptoethanol as reducing agent (2-MCE). Additionally sulfhydryl groups are blocked with iodoacetic acid. Separation is carried out at a flow rate of 1 ml/min using 40 mM NaH₂PO₄ (eluent A, pH=7.8, adjusted with NaOH) as polar and a methanol water mixture (100/l) as non-polar phase (eluent B). The following gradient is applied: Start 0% B; 39 min 39% B; 70 min 64% B; 100% B for 3.5 min; 2 min 0% B for equilibration. Derivatization at room temperature is automated as described below. Initially 0.5 μl of 0.5% 2-MCE in bicine (0.5M, pH 8.5) are mixed with 0.5 μl cell extract. Subsequently 1.5 μl of 50 mg/ml iodoacetic acid in bicine (0.5M, pH 8.5) are added, followed by addition of 2.5 μl bicine buffer (0.5M, pH 8.5). Derivaiization is done by adding 0.5 μl of 10 mg/ml OPA reagent dissolved in 1/45/54 v/v/v of 2-MCE/MeOH/bicine (0.5M, pH 8.5). Finally the mixture is diluted with 32 μl H₂O. Between each of the above pipetting steps there is a waiting time of 1 min. A total volume of 37.5 μl is then injected onto the column. Note, that the analytical results can be significantly improved, if the auto sampler needle is periodically cleaned during (e.g. within waiting time) and after sample preparation. Detection is performed by a fluorescence detector (340 nm excitation, emission 450 nm, Agilent, Waldbronn, Germany). For quantification α-amino butyric acid (ABA) is used as internal standard

Definition of Recombination Protocol

In the following it will be described how a strain of C. glutamicum with increased efficiency of methionine production can be constructed implementing the findings of the above predictions. Before the construction of the strain is described, a definition of a recombination event/protocol is given that will be used in the following.

“Campbell in,” as used herein, refers to a transformant of an original host cell in which an entire circular double stranded DNA molecule (for example a plasmid being based on pCLIK int sacB has integrated into a chromosome by a single homologous recombination event (a cross-in event), and that effectively results in the insertion of a linearized version of said circular DNA molecule info a first DNA sequence of the chromosome that is homologous to a first DNA sequence of the said circular DNA molecule. “Campbelled in” refers to the linearized DNA sequence that has been integrated into the chromosome of a “Campbell in” transformant. A “Campbell in” contains a duplication of the first homologous DNA sequence, each copy of which includes and surrounds a copy of the homologous recombination crossover point. The name comes from Professor Alan Campbell, who first proposed this kind of recombination.

“Campbell out,” as used herein, refers to a cell descending from a “Campbell in” transformant, in which a second homologous recombination event (a cross out event) has occurred between a second DNA sequence that is contained on the linearized inserted DNA of the “Campbelled in” DNA, and a second DNA sequence of chromosomal origin, which is homologous to the second DNA sequence of said linearized insert, the second recombination event resulting in the deletion (jettisoning) of a portion of the integrated DNA sequence, but, importantly, also resulting in a portion (this can be as little as a single base) of the integrated Campbelled in DNA remaining in the chromosome, such that compared to the original host cell, the “Campbell out” cell contains one or more intentional changes in the chromosome (for example, a single base substitution, multiple base substitutions, insertion of a heterologous gene or DNA sequence, insertion of an additional copy or copies of a homologous gene or a modified homologous gene, or insertion of a DNA sequence comprising more than one of these aforementioned examples listed above).

A “Campbell out” cell or strain is usually, but not necessarily, obtained by a counter-selection against a gene that is contained in a portion (the portion that is desired to be jettisoned) of the “Campbelled in” DNA sequence, for example the Bacillus subtilis sacB gene, which is lethal when expressed in a cell that is grown in the presence of about 5% to 10% sucrose. Either with or without a counter-selection, a desired “Campbell out” ceil can be obtained or identified by screening for the desired cell, using any screenable phenotype, such as, but not limited to, colony morphology, colony color, presence or absence of antibiotic resistance, presence or absence of a given DNA sequence by polymerase chain reaction, presence or absence of an auxotrophy, presence or absence of an enzyme, colony nucleic acid hybridization, antibody screening, etc. The term “Campbell in” and “Campbell out” can also be used as verbs in various tenses to refer to the method or process described above.

It is understood that the homologous recombination events that leads to a “Campbell in” or “Campbell out” can occur over a range of DNA bases within the homologous DNA sequence, and since the homologous sequences will be identical to each other for at least part of this range, it is not usually possible to specify exactly where the crossover event occurred. In other words, it is not possible to specify precisely which sequence was originally from the inserted DNA, and which was originally from the chromosomal DNA. Moreover, the first homologous DNA sequence and the second homologous DNA sequence are usually separated by a region of partial non-homology, and it is this region of non-homology that remains deposited in a chromosome of the “Campbell out” cell.

For practicality, in C. glutamicum, typical first and second homologous DNA sequence are at least about 200 base pairs in length, and can be up to several thousand base pairs in length, however, the procedure can be made to work with shorter or longer sequences. For example, a length for the first and second homologous sequences can range from about 500 to 2000 bases, and the obtaining of a “Campbell out” from a “Campbell in” is facilitated by arranging the first and second homologous sequences to he approximately the same length, preferably with a difference of less than 200 base pairs and most preferably with the shorter of the two being at least 70% of the length of the longer in base pairs, The “Campbell In and-Out-method” is described in WO2007012078

The invention will now be illustrated by means of various examples. These examples are however in no way meant to limit the invention in any way.

### Examples

In the following it will be shown how the codon usage of abundant proteins in C. glutamicum was identified. Furthermore, examples are presented which show that usage of modified nucleotide sequences which have been optimized with regard to either the codon usage of abundant proteins or the organism of C. glutamicum can be used to either increase or reduce the amount of a protein in C. glutamicum. This is shown for foreign genes as well as endogenous genes.

1. Identification of Abundant Proteins in C. glutamicum

Cellular extracts were prepared from the C. glutamicum strain ATCC 13032 and of some derivatives. For tins purpose, 250 mg of cell grown under standard conditions were pelleted and suspended in 750 μl lysis huffier (20 mM TRIS, 5 mM EDTA, phi 7.5) containing a protease inhibitor mix (Complete, Roche) Cell disruption was carried out at 4° C. in a mixer mil) (Retseh, MM 2000) using 0.25-0.5 mm glass beads. Cell debris was removed by centrifugation at 22.000 rpm for 1 hour at 4° C. Protein concentrations were determined by the Popov (Popov et a. (1975) Acta. Biol. Med. Germ, 34, 1441-1446). Cell extracts were used immediately or frozen in aliquots at −80° C.

For 2D polyacrylamide gel electrophoresis of proteins 30 μg of crude protein extract was resuspended 450 μl of rehydration buffer (8M urea, 2M thiourea, 1% CHAPS, 20 mM DTT, 1% Ampholines 3.5-10) and a few grains of bromophenol blue. For isoelectric focussing precast 24 cm-IPG strips with a linear pH gradient of 4.5 to 5.5 were used in a Multiplier II isoelectric focussing unit (Amersham Biosciences). Proteins were focused using a gradient programme up to 3500 V resulting 65.000 Vh in total. Focused IPG gels were equilibrated twice for 15 minutes in a buffer containing 1.5 M Tris-HCl (pH 8.8), 6M urea, 30% (vol/vol) glycerol, 2% (wt/vol) sodium dodecyl sulfate, and 1% (wt/vol) DTT. For the second equilibration step DDT was replaced by 5% (wt/vol) iodoacetamide, and a few grains of bromophenol blue were added. The second dimension was run in sodium dodecyl sulfate-12.5% polyacrylamide gels in an Ettan Dalt apparatus (Amersham Biosciences) as recommended by the manufacturer, and gels were subsequently silver stained (Blum et al. (1987), Electrophoresis, 8, 93-99) in a home made staining automat.

Protein spots were excised from preparative Coomassie-stained gels (300 μg total protein load each) and digested with modified trypsin (Roche, Mannheim) as described by Hermann et al. (Electrophoresis (2001), 22, 1712-1723). Mass spectrometrical identifications were performed on an LCQ advantage (Thermo Electron) after nano-HPLC separation of the peptides (LC Packings, RP18 column, length 15 cm, i.d. 75 μm), using the MASCOT software (David et al. (1999) Electrophoresis, 20, 3551-3567).

Based on the 2D gel electrophoresis results of different gels 14 proteins were identified as being abundant in C. glutamicum as these proteins could be observed at elevated amounts in all gels. These proteins are: Elongation Factor Tu (Genbank accession no: X77034), glycerine-aldehyde-3-phosphate-dehydrogenase (Genbank accession no: BX927152, ±, nt. 289401-288397), fructose bisphosphate aldolase (Genbank accession no: BX927156, ±, nt. 134992-133958), Elongation Factor Ts (Genbank accession no: BX927154, ±, nt. 14902-14075), hypothetical protein (Genbank accession no: BX927155, ±, nt. 213489-214325) enolase (Genbank accession no: BX927150, nt. 338561-339838) peptidyl-prolyl cis-trans isomerase (Genbank accession no: BX927148, nt. 34330-34902), superoxide dismutase (Genbank accession no: AB055218) phospho-glycerate dehydrogenase (Genbank accession no: BX927151, nt. 306039-307631) SSU Rib protein S1P (Genbank accession no: BX927152, ±, nt. 26874-28334) triosephosphate-isomerase (Genbank accession no: BX927152, ±, nt. 286884-286105) isopropyl malate synthase (Genbank accession no: X70959) butan-2,3-dioldehydrogenase (Genbank accession no: BX927156, nt. 20798-21574) and fumarate hydratase (Genbank accession no: BX927151, ±, nt. 18803-17394).

The coding sequences of these genes were then fed into the “Cusp” function of the EMBOSS tool box using standard parameters in an independent approach the genomic sequence of the complete C. glutamicum strain ATCC 13032 was used to generate a codon usage table for the organism as a whole.

The codon usage frequencies as determined for the aforementioned 14 abundant proteins were used to calculate codon usage frequencies for abundant proteins in C. glutamicum. The codon relative codon usage frequencies of abundant proteins in C. glutamicum are found in table 2, while the relative codon usage frequencies of the organism as a whole are found in table 1.

TABLE 1
Relative codon usage frequencies of
Corynebacterium glutamicum ATCC 13032.

| | | | | | | | |
|---|---|---|---|---|---|---|---|
| UUU | 37.1 | UCU | 17.3 | UAU | 33.8 | UGU | 36.5 |
| UUC | 62.9 | UCC | 33.6 | UAC | 66.2 | UGC | 63.5 |
| UUA | 5.3 | UCA | 13.0 | UAA | 53.1 | UGA | 16.7 |
| UUG | 20.3 | UCG | 11.9 | UAG | 30.2 | UGG | 100 |
| CUU | 17.2 | CCU | 23.3 | CAU | 32.1 | CGU | 24.5 |
| CUC | 22.5 | CCC | 20.2 | CAC | 67.9 | CGC | 44.7 |
| CUA | 6.1 | CCA | 34.9 | CAA | 38.5 | CGA | 11.8 |
| CUG | 28.6 | CCG | 21.6 | CAG | 61.5 | CGG | 8.8 |
| AUU | 37.7 | ACU | 20.4 | AAU | 33.4 | AGU | 7.8 |
| AUC | 59.2 | ACC | 52.9 | AAC | 66.4 | AGC | 16.4 |
| AUA | 3.1 | ACA | 12.5 | AAA | 39.9 | AGA | 4.1 |
| AUG | 100 | ACG | 14.2 | AAG | 60.1 | AGG | 6.1 |
| GUU | 26.0 | GCU | 23.7 | GAU | 55.6 | GGU | 30.3 |
| GUC | 27.7 | GCC | 25.4 | GAC | 44.4 | GGC | 42.4 |
| GUA | 10.1 | GCA | 29.3 | GAA | 56.3 | GGA | 18.9 |
| GUG | 36.2 | GCG | 21.6 | GAG | 43.7 | GGG | 8.4 |
| ATG* | 72.5 | | | | | | |
| GTG* | 20.5 | | | | | | |
| TTG* | 7.0 | | | | | | |

*designates start codons; relative Frequencies are in percentage.

TABLE 2
Relative codon usage frequencies of 14 abundant
proteins in Corynebacterium glutamicum ATCC 13032.

| | | | | | | | |
|---|---|---|---|---|---|---|---|
| UUU | 10.6 | UCU | 20.2 | UAU | 3.6 | UGU | 25.0 |
| UUC | 89.4 | UCC | 65.4 | UAC | 96.4 | UGC | 75.0 |
| UUA | 0.8 | UCA | 3.3 | UAA | 92.9 | UGA | 0.0 |
| UUG | 7.5 | UCG | 2.2 | UAG | 7.1 | UGG | 100 |
| CUU | 20.8 | CCU | 37.6 | CAU | 5.8 | CGU | 39.6 |
| CUC | 25.4 | CCC | 4.4 | CAC | 94.2 | CGC | 57.6 |
| CUA | 2.6 | CCA | 51.4 | CAA | 7.7 | CGA | 2.2 |
| CUG | 42.9 | CCG | 6.6 | CAG | 92.3 | CGG | 0.6 |
| AUU | 17.1 | ACU | 18.9 | AAU | 9.7 | AGU | 0.8 |
| AUC | 82.6 | ACC | 78.9 | AAC | 90.3 | AGC | 8.1 |
| AUA | 0.3 | ACA | 1.4 | AAA | 7.1 | AGA | 0.0 |
| AUG | 100 | ACG | 0.8 | AAG | 92.9 | AGG | 0.0 |
| GUU | 47.9 | GCU | 46.8 | GAU | 34.9 | GGU | 32.3 |
| GUC | 34.0 | GCC | 9.9 | GAC | 65.1 | GGC | 59.0 |
| GUA | 6.5 | GCA | 35.9 | GAA | 32.5 | GGA | 8.2 |
| GUG | 11.6 | GCG | 7.4 | GAG | 67.5 | GGG | 0.5 |
| ATG* | 78.6 | | | | | | |
| GTG* | 21.4 | | | | | | |
| TTG* | 0.0 | | | | | | |

*indicates start codons; relative Frequencies are in percentage.

Table 1 was then used to determine the codons that are least frequently for each amino acid in C. glutamicum. This information is displayed in table 3 below.

TABLE 3
The least frequently used codons in
Corynebacterium glutamicum ATCC 13032.

| | |
|---|---|
| UUU | F |
| AGU | S |
| UAU | Y |
| UGU | C |
| UGA | Stop |
| AUG | M |
| UGG | W |
| UUA | L |
| CCC | P |
| CAU | H |
| AGA | R |
| CAA | Q |
| AUA | I |
| ACA | T |
| AAU | N |
| TTG (if start codon) | M |
| AAA | K |
| GUA | V |
| GCG | A |
| GAC | D |
| GGG | G |
| GAG | E |

Table 2 was then used to determine the codons that are least frequently for each amino acid in abundant proteins of C. glutamicum. This information is displayed in table 4 below.

TABLE 4
The least frequently used codons in abundant
proteins of Corynebacterium glutamicum ATCC 13032.

| | |
|---|---|
| UUU | F |
| AGU | S |
| UAU | Y |
| UGU | C |
| UGA | Stop |
| AUG | M |
| UGG | W |
| UUA | L |
| CCC | P |
| CAU | H |
| AGA, AGG | R |
| CAA | Q |
| AUA | I |
| ACG | T |
| AAU | N |
| TTG (if start codon) | M |
| AAA | K |
| GUA | V |
| GCG | A |
| GAU | D |
| GGG | G |
| GAA | E |

Table 5 shows the frequencies of codons which are not calculated on the basis of codons encoding a specific amino acid, but on the basis of all codons for all amino acids. The values in brackets indicate the absolute number of the respective codon. The relative frequencies of table 1 were calculated on the basis of these absolute numbers. The values refer to the genome of C. glutamicum.

TABLE 5
Codon usage of Corynebacterium glutamicum ATCC 13032.

| | | | | | | | | | | | |
|---|---|---|---|---|---|---|---|---|---|---|---|
| UUU | 13.4 | (25821) | UCU | 11.0 | (21227) | UAU | 7.5 | (14384) | UGU | 2.4 | (4605) |
| UUC | 22.8 | (43837) | UCC | 21.4 | (41118) | UAC | 14.7 | (28214) | UGC | 4.2 | (8015) |
| UUA | 5.1 | (9795) | UCA | 8.3 | (15898) | UAA | 1.7 | (3272) | UGA | 0.5 | (1032) |
| UUG | 19.6 | (37762) | UCG | 7.6 | (14639) | UAG | 1.0 | (1859) | UGG | 14.1 | (27072) |
| CUU | 16.7 | (32074) | CCU | 11.3 | (21668) | CAU | 6.8 | (12991) | CGU | 13.7 | (26310) |
| CUC | 21.8 | (41988) | CCC | 9.7 | (18716) | CAC | 14.3 | (27445) | CGC | 24.9 | (47939) |
| CUA | 5.9 | (11320) | CCA | 16.9 | (32429) | CAA | 13.0 | (24975) | CGA | 6.6 | (12698) |
| CUG | 27.7 | (53261) | CCG | 10.4 | (20070) | CAG | 20.7 | (39864) | CGG | 4.9 | (9466) |
| AUU | 21.7 | (41804) | ACU | 12.6 | (24184) | AAU | 10.9 | (21056) | AGU | 4.9 | (9515) |
| AUC | 34.1 | (65557) | ACC | 32.5 | (62592) | AAC | 21.8 | (42037) | AGC | 10.4 | (20019) |
| AUA | 1.8 | (3483) | ACA | 7.7 | (14747) | AAA | 13.9 | (26703) | AGA | 2.3 | (4445) |
| AUG | 22.1 | (42484) | ACG | 8.8 | (16879) | AAG | 20.9 | (40213) | AGG | 3.3 | (6398) |
| GUU | 20.8 | (40069) | GCU | 25.4 | (48864) | GAU | 33.0 | (63429) | GGU | 24.3 | (46678) |
| GUC | 22.2 | (42696) | GCC | 27.2 | (52264) | GAC | 26.4 | (50716) | GGC | 34.0 | (65427) |
| GUA | 8.1 | (15628) | GCA | 31.3 | (60329) | GAA | 35.7 | (68737) | GGA | 15.2 | (29219) |
| GUG | 28.9 | (55708) | GCG | 23.2 | (44613) | GAG | 27.7 | (53381) | GGG | 6.7 | (12923) |

Frequencies are indicated after the codons in /1000.

Table 6 shows the frequencies of codons which were not calculated on the basis of codons encoding a specific amino acid, but on the basis of all codons for all amino acids. The values in brackets indicate the absolute number of the respective codon. The relative frequencies of table 2 were calculated on the basis of these absolute numbers. The values refer to the group of abundant proteins in C. glutamicum.

TABLE 6

Codon usage of 14 abundant proteins in
Corynebacterium glutamicum ATCC 13032.

| | | | | | | | | | | | |
|---|---|---|---|---|---|---|---|---|---|---|---|
| UUU | 3.6 | (18) | UCU | 10.9 | (55) | UAU | 0.8 | (4) | UGU | 1.2 | (6) |
| UUC | 30.0 | (152) | UCC | 35.2 | (178) | UAC | 21.1 | (107) | UGC | 3.6 | (18) |
| UUA | 0.6 | (3) | UCA | 1.8 | (9) | UAA | 2.6 | (13) | UCA | 0.0 | (0) |
| UUG | 5.7 | (29) | UCG | 1.2 | (6) | UAG | 0.2 | (1) | UGG | 8.3 | (42) |
| CUU | 16.0 | (81) | CCU | 13.4 | (68) | CAU | 1.2 | (6) | CGU | 17.4 | (88) |
| CUC | 19.6 | (99) | CCC | 1.6 | (8) | CAC | 19.4 | (98) | CGC | 25.3 | (128) |
| CUA | 2.0 | (10) | CCA | 18.4 | (93) | CAA | 2.6 | (13) | CGA | 1.0 | (5) |
| CUG | 33.0 | (167) | CCG | 2.4 | (12) | CAG | 30.6 | (155) | CGG | 0.2 | (1) |
| AUU | 9.9 | (50) | ACU | 10.5 | (53) | AAU | 4.0 | (20) | AGU | 0.4 | (2) |
| AUC | 47.8 | (242) | ACC | 43.7 | (221) | AAC | 36.8 | (186) | AGC | 4.3 | (22) |
| AUA | 0.2 | (1) | ACA | 0.8 | (4) | AAA | 3.6 | (18) | AGA | 0.0 | (0) |
| AUG | 17.2 | (87) | ACG | 0.4 | (2) | AAG | 46.4 | (235) | AGG | 0.0 | (0) |
| GUU | 43.5 | (220) | GCU | 54.9 | (278) | GAU | 21.9 | (111) | GGU | 28.1 | (142) |
| GUC | 30.8 | (156) | GCC | 11.7 | (59) | GAC | 40.9 | (207) | GGC | 51.2 | (259) |
| GUA | 5.9 | (30) | GCA | 42.1 | (213) | GAA | 27.9 | (141) | GGA | 7.14 (36) | |
| GUG | 10.5 | (53) | GCG | 8.7 | (44) | GAG | 57.9 | (293) | GGG | 0.4 | (2) |

Frequencies are indicated after the codons in /1000.

Surprisingly there are many significant differences in the codon usage between tables generated by using all proteins (whole genome, table 1) compared to the situation where only the above-specified abundant genes are considered (table 2). Some of the examples are shown in table 7 below.

TABLE 7
Relative frequency of codons used

| Amino acid | Codon | Whole genome | Abundant proteins |
|---|---|---|---|
| q | caa | 38.5% | 7.7 |
| q | cag | 61.5% | 91.3 |
| y | tac | 66.0% | 96.4 |
| y | tat | 33.8% | 3.6 |

3. Reduction of Expression of an Endogenous Protein

It has been set out above that codon usage modification may be used to decrease protein expression. This will he illustrated in the following for the proteins isocitrate dehydrogenase (icd) and diaminopimelate synthase (dapA).

3.1 Reducing Expression of Isocitrate Dehydrogenase (icd)

Cloning

To reduce the activity of the isocitrate dehydrogenase (Genbank Accession code X71489), two different changes in codon usage were made. In all cases the codons of the coding sequence were changed without changing the amino acid sequence of the encoded protein. The manipulations were ah made on the only chromosomal copy of the icd gene of Corynebacterium glutamicum. The subsequent measurement of ICD activity directly allows a readout of the effect, as one can assume that it reflects the expression level given that the enzyme itself is not changed. The modifications are shown in table 8.

TABLE 8
Overview codon exchanges in ICD

| | | | affected amino |
|---|---|---|---|
| | name | Description | acid positions |
| 1 | ICD ATG → GTG | Change of the start codon | 1 (Met) |
| | | from ATG to GTG | |
| 2 | ICD CA2 | Change of a glycine and an | 32 (Gly), 33 (Ile) |
| | | isoleucine codon from GGC | |
| | | ATT to GGG ATA | |

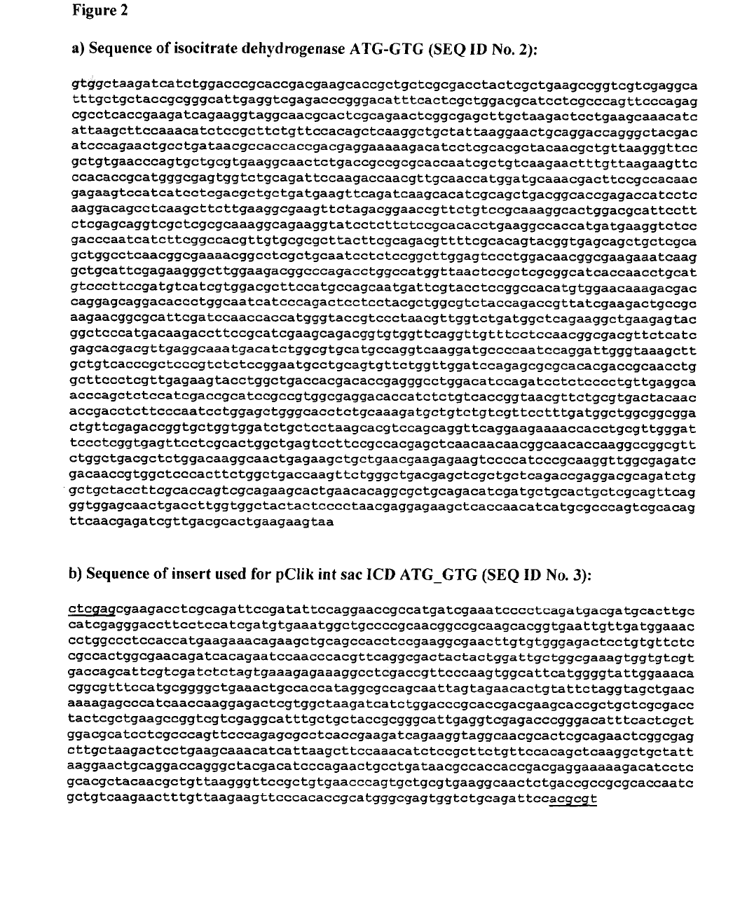

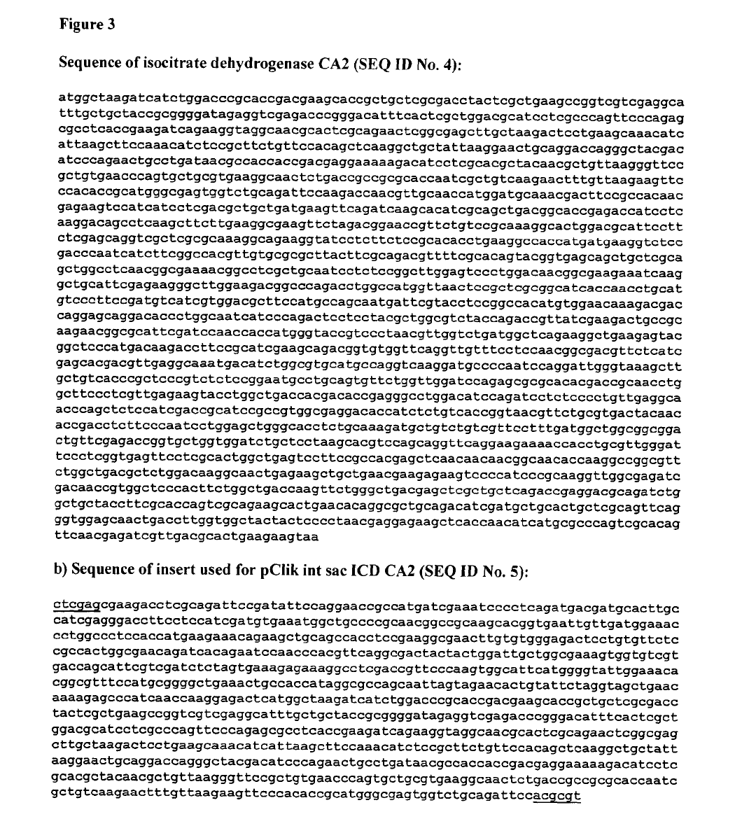

The sequence of ICD ATG-GTG is depicted in FIG. 2a). The sequence of ICD CA is depicted in FIG. 3a). To introduce these mutations into the chromosomal copy of the icd coding region, 2 different plasmids were constructed which allow the marker-free manipulation by 2 consecutive homologous recombination events.

To this end the sequences of ICD ATG-GTG and ICD CA2 were cloned into the vector pClik int sacB (Becker et al (2005), Applied and Environmental Microbiology, 71 (12), p. 8587-8596) being a plasmid containing the following elements:
Kanamycin-resistance gene
SacB-gene which can be used as a positive selection marker as cells which carry this gene cannot grow on sucrose containing medium.
Origin of replication for E. coli
Multiple Cloning Site (MCS)

This plasmid allows the integration of sequences at the genomic locus of C. glutamicum.

Construction of the Plasmids

All inserts were amplified by PCR using genomic DNA of ATCC 13032 as a template. The modification of the coding region was achieved by fusion PCR using the following oligonucleotides: The table shows the primers used as well as the template DNA

TABLE 9
Overview of primers for cloning idh constructs

| | | | Fusion | |
|---|---|---|---|---|
| | PCR A | PCR B | PCR | |
| ICD ATG → | Old 441 | Old 443 | Old 441 | Primer 1 |
| GTG | Old 444 | Old 442 | Old 442 | Primer 2 |
| | Genom. DNA | Genom. DNA | PCR A + B | Template |
| | of | of | | |
| | ATCC 13032 | ATCC 13032 | | |
| ICD CA2 | Old 441 | Old 447 | Old 441 | Primer 1 |
| | Old 448 | Old 442 | Old 442 | Primer 2 |
| | Gemome. DNA | Genom. DNA | PCR A + B | Template |
| | of | of | | |
| | ATCC 13032 | ATCC 13032 | | |

Old 441 GAGTACCTCGAGCGAAGACCTCGCAGATTCCG (SEQ ID No. 6)
Old 442 CATGAGACGCGTGGAATCTGCAGACCACTCGC (SEQ ID No. 7)
Old 443 GAGACTCGTGGCTAAGATCATCTG (SEQ ID No. 8)
Old 444 CAGATGATCTTAGCCACGAGTCTC (SEQ ID No. 9)
Old 447 CTACCGCGGGGATAGAGG (SEQ ID No. 10)
Old 443 CCTCTATCCCCGCGGTAG (SEQ ID No. 11)

In all cases the product of the fusion PCR was purified, digested with XhoI and MluI, purified again and ligated into pClik int sacB which had been linearized with the same restriction enzymes. The integrity of the insert was confirmed by sequencing.

The coding sequence of the optimised sequence ICD ATG→GTG is shown in FIG. 2 (SEQ ID 2). The coding sequence of the optimised sequence ICD CA2 is shown in FIG. 3 (SEQ ID 4).

Construction of Strains with Modified ICD Expression Levels

The plasmids were then used to replace the native coding region of these genes by the coding regions with the modified coding usage. The strain used was ATCC 13032 lysC fbr.

Two consecutive recombination events one in each of the up- and the downstream region respectively are necessary to change the complete coding sequence. The method of replacing the endogenous genes with the optimized genes is in principle described in the publication by Becker et ah (vide supra). The most important steps are:
Introduction of the plasmids in the strain by electroporation. The step is e.g. described in DE 10046870 which is incorporated by reference as far as introduction of plasmids into strains is disclosed therein,
Selection of clones that have successfully integrated the plasmid after a first homologous recombination event into the genome. This selection is achieved by growth on kanamycine-containing agar plates. In addition to that selection step, successful recombination can be checked via colony PCR.
   Primers used to confirm the presence of the plasmid in the genome were:
   BK1776 (AACGGCAGGTATATGTGATG) (SEQ ID No. 12) and OLD 450(CGAGTAGGTCGCGAGCAG) (SEQ ID No. 13). The positive clones give a band of ca. 600 bp)
By incubating a positive clone in a kanamycine-free medium a second recombination event is allowed for.
Clones in which the vector backbone has been successfully removed by way of a second recombination event are identified by growth on sucrose-containing medium. Only those clones will survive that have lost the vector backbone comprising the SacB gene.
Then, clones in which the two recombination events have led to successful replacement of the native idh-coding region were identified by sequencing of a PCR-product spanning the relevant region. The PCR-product was generated using, genomic DNA of individual clones as a template and primers OLD 441 and OLD 442. The PCR-product was purified and sequenced with Old 471 (GAATCCAACCCACGTTCAGGC) (SEQ ID No. 14)

One may use different C. glutamicum strains for replacing the endogenous copy of ICD. However, it is preferred to use a C. glutamicum lysine production strain such as for example ATCC13032 lysC^{fbr} or other derivatives of ATCC13032 or ATCC13286

ATCC13032 lysC^{fbr} may be produced starting from ATCC13032. In order to generate such a lysine producing strain, an allelic exchange of the lysC wild type gene was performed in C. glutamicum ATCC13032. To this end a nucleotide exchange was introduced into the lysC gene such that the resulting protein carries an isoleucine at position 311 instead of threonine. The detailed construction of this strain is described in patent application WO2005059093. The accession no. of the lysC gene is P26512.

To analyze the effect of the codon usage amended IDH ATG-GTG and IDH CA2, the optimized strains are compared to lysine productivity of the parent strain.

Determination of ICD Activity

One to two clones of each mutant strain were tested for ICD activity. Cells were grown in liquid culture over night at 30° C., harvested in exponential growth phase by centrifugation. The cells were washed twice with 50 mM Tris-HCl, pH 7.0, 200 mg cells were resuspended in 800 μl lysis buffer (50 mM Tris-HCL, pH 7.0, 10 mM MgCl2, 1 mM: DTT, 10% Glycerol) and disrupted by bead beating (Ribolyser, 2×30 s, intensity 6). The cell debris was pelleted by centrifugation (table top centrifuge, 30 min, 13 K). The resulting supernatant is an extract of soluble proteins which was used as the following enzyme assay.

ICD activity was monitored by increase of absorption at 340 nm due to the reduction of NADP in a total volume of 1 ml under the following conditions:
30 mM Triethanolamine-chloride, pH 7.4, 0.4 mM NADP, 8 mM DL-Isocitrate, 2 mM MnSO4, cell lysate corresponding to 0.1-0.2 mg protein

ICD activities were calculated using the molar extinction coefficient of 6.22/mM*cm for NADPH.

Results

The measured ICD activities were as follows:

TABLE 10
ICD activity

| | | Specific activity of cell extract |
|---|---|---|
| Strain | Clone | U*μmol/ml*min*mg protein |
| ATCC lysC fbr | | 0.29 |
| ATCC lysC fbr | | 0.26 |
| ICD ATG → GTG | | 0.04 |
| ICD CA2 | 1 | 0.10 |
| ICD CA2 | 2 | 0.10 |

Effect on Lysine Productivity

To analyze the effect of the modified expression of ICD on lysine productivity, the optimized strains are compared to lysine productivity of the parent strains.

To this end one the strains were grown on CM-plates (10% sucrose, 10 g/l glucose, 2.5 g/l NaCl, 2 g/l urea, 10 g/l Bacto Pepton, 10 g/l yeast extract, 22 g/l agar) for 2 days at 30° C. Subsequently cells were scraped from the plates and re-suspended in saline. For the main culture 10 ml of medium 1 and 0.5 g autoclave CaCo₃ in a 100 ml Erlenmeyer flask were incubated together with the cell suspension up to an OD₆₀₀ of 1.5. The cells were then grown for 72 hours on a shaker of the type Infors AJ118 (Infors, Bottmingen, Switzerland) at 220 rpm. Medium 1 had the same concentration as mentioned in experiment 3.3:

Subsequently, the concentration of lysine that is segregated into the medium was determined. This was dome using HPLC on an Agilent 1100 Series LC system HPLC. A precolumn derivatisation with ortho-phthalaldehyde allowed to quantify the formed amino acid. The separation of the amino acid mixture can be done on a Hypersil AA-column (Agilent).

The determined lysine concentration values shown are average data from 2 independent cultivations. The deviations from the average was always below 4%

TABLE 11
Lysine productivity

| | | Relative lysine amount | Relative OD |
|---|---|---|---|
| Strain | Clone | [%] | [%] |
| ATCC lysC fbr | | 100.00 | 100.00 |
| ATCC lysC fbr | | 99.81 | 101.22 |
| ICD ATG → GTG | | 102.34 | 92.77 |
| ICD CA2 | 1 | 101.44 | 99.80 |
| ICD CA2 | 2 | 104.85 | 96.23 |

It can be easily seen that strains with lowered ICD activity have higher lysine productivities. As all carbon source is used after 72 h, one can also directly see that the carbon yield (amount of formed product per sugar consumed) is higher in these strains.

The amount of other amino acids in the medium after another cultivation was also analyzed. Interestingly, the amount of glycine is also influenced by ICD expression.

The table shows the amount of glycine obtained alter 48 h of cultivation. Each clone was cultivated in three independent flasks.

TABLE 12
Glycine productivity

| | | Glycine |
|---|---|---|
| | | (μmol/l |
| Medium | | 5.72 |
| ATCC lysC fbr | | 78.56 |
| | | 81.33 |
| | | 79.40 |
| ATCC lysC fbr | | 86.29 |
| | | 78.51 |
| | | 81.35 |
| ICD ATG → GTG | | 110.44 |
| | | 108.51 |
| | | 117.36 |
| ICD CA2 | 1 | 103.29 |
| | | 104.56 |
| | | 96.26 |
| ICD CA2 | 2 | 98.83 |
| | | 98.72 |
| | | 99.39 |

It can be seen that strains with lower ICD activity accumulate higher amounts of glycine,

In a further experiment isocitrate dehydrogenase carrying the above mentioned ATG-GTG mutation in the start codon was cloned into pClik as described above leading to pClik int sacB ICD (ATG-GTG) (SEQ ID No. 15). Subsequently, strain M2620 was constructed by campbelling in and campbelling out the plasmid pClik int sacB ICDH (ATG-GTG) (SEQ ID No. 15) into genome of the strain OM469. The strain OM469 has been described in WO2007012078.

The strain was grown as described in WO2007020295. After 48 h incubation at 30° C. the samples were analyzed for sugar consumption. It was found that the strains had used up all added sugar, meaning that all strains had used the same amount of carbon source. Synthesized methionine was determined by HPLC as described above and in WO2007020295.

TABLE 13
Methionine production

| Strain | Methionine (mM) |
|---|---|
| OM 469 | 10.2 |
| M2620 | 23.7 |

From the data in table 13 it can be seen that the strain M2620 with an altered start codon of the ICDH gene and therefore altered ICDH activity has higher methionine productivity. Since all carbon source is used up after 48 h, one can also directly see, that the carbon yield (amount of formed product per sugar consumed) for the produced methionine is higher in this strain.

3.2. Reduced Protein Expression of dapA in C. glutamicum

In the following, it is described how to decrease the amount of dapA by adapting the codon usage as mentioned above.

The enzyme dihydropicolinate synthase (dapA) is important for lysine biosynthesis. The wild type sequence of C. glutamicum dapA is given as SEQ ID No. 16. The codon usage of the coding sequence of dapA was determined using the Cusp function of the EMBOSS software package.

Then, the bases corresponding to positions 12 (T to G), 27 (C to A), 30 (A to G), 45 (C to G) and 54 (A to G) were altered using established mutagenesis methods (Quikchange kit, Stratagene La Jolla USA) resulting in altered codons which still coded for the same amino acids (SEQ ID No. 17).

The mutated gene sequence was cloned into a vector for chromosomal integration as mentioned in WO2007020295.

Briefly, The strain M2059 was constructed by campbelling in and out the plasmid pK19PSOD ask (SEQ ID No. 18) into the strain. OM99 to overexpress the aspartokinase gene The strain M2121 was constructed by campbelling in and out the plasmid pCLIK Int Psod Glucose-6-PDH (SEQ ID No. 19) into the strain M2059 to over express glucose-6-phosphate dehydrogenase gene (zwf gene). The strain M2199 was constructed by campbelling in and out the plasmid pK19 sacB Glu-6-P-DH (SEQ ID No. 20) replacement into the strain M2121 to express a glucose-6-phosphate dehydrogenase mutant gene (zwf gene). The strain M2271 was constructed by campbelling in and out the plasmid pK19 PGro PycA (SEQ ID No. 21) into the strain M2199 to express a mutated pycA gene. The strain M2376 was constructed by campbelling in and out the plasmid pCLIK int sacB dapA codon low (SEQ ID No. 22) into the strain M2271 to express the mutated dapA.

The resulting strain was named M2376 and grown as described in 2007020295. Aminoacids were determined after 48 h incubation and are described as mM concentration (see table 14).

TABLE 14
lysine and homoserine production

| Strain | homoserine (mM) |
|---|---|
| M2271 | 78 |
| M2376 (=M2271 + | 103 |
| dapA mutated) | |

It was found that strains expressing the mutated dapA gene showed an increased accumulation of homoserine when, compared to the isogenic strain showing that the activity of dapA was decreased over the unmutated version of dapA

1.-28. (canceled)

29. A method of reducing the amount of at least one polypeptide in a host cell, comprising expressing in a host cell a modified nucleotide sequence instead of a non-modified nucleotide sequence encoding for a polypeptide of the same amino acid sequence and function, wherein said modified nucleotide sequence is derived from the non-modified nucleotide sequence such that at least one codon of the non-modified nucleotide sequence is replaced in the modified nucleotide sequence by a less frequently used codon according to the codon usage of the host cell.

30. The method of claim 29, wherein the host cell is selected from microorganisms, insect cells, plant cells or mammalian cell culture systems.

31. The method of claim 29, wherein the host cell is a Corynebacterium, and wherein the modified nucleotide sequence uses for each replaced ammo acid the least frequently used codon.

32. The method of claim 31, wherein all codons of said modified nucleotide sequence for each amino acid are selected from the codon usage of table 3.

33. The method of claim 31, wherein the Corynebacterium is C. glutamicum.

34. The method of claim 31, wherein the Corynebacterium is C. glutamicum strain ATCC 13032 or derivatives thereof.

35. The method of claim 29, wherein the method is used to decrease expression of said polypeptide in the host cell by at least 5%, 10%, 20%, 30%, 40%, 50%, 60%, 70%, 80%, 90% or 95%, with the extent, of reduction of expression being determined in comparison to the level of expression of the polypeptide that is expressed from the non-modified nucleotide sequence under comparable conditions.

36. A recombinant modified nucleotide sequence encoding for a polypeptide which allows for reduced expression of said polypeptide in a host cell wherein the modified nucleotide sequence is derived from a non-modified nucleotide sequence which encodes for a polypeptide of the same amino acid sequence and function wherein said modified nucleotide sequence is derived from the endogenous nucleotide sequence such that at least one codon of the non-modified nucleotide sequence is replaced in the modified nucleotide sequence by a less frequently used codon according to the codon usage of the host cell, wherein the modified nucleotide sequence uses for each replaced amino acid the least frequently used codon.

37. The recombinant modified nucleotide sequence of claim 36, wherein at least one codon of the non-modified nucleotide sequence is replaced in the modified nucleotide sequence by the least frequently used codon with codon frequency being determined for Corynebacterium.

38. The recombinant modified nucleotide sequence of claim 37, wherein the Corynebacterium is C. glutamicum.

39. The recombinant modified nucleotide sequence of claim 37, wherein all codons of said modified nucleotide sequence for each amino acid are selected from table 3.

40. The recombinant modified nucleotide sequence of claim 36, wherein said non-modified nucleotide sequence is selected from the group comprising nucleotide sequences encoding genes of biosynthetic pathways of fine chemicals.

41. The recombinant modified nucleotide sequence of claim 40, wherein the fine chemicals comprise amino acids, alcohols, monomers for polymer synthesis, sugars, lipids, oils, fatty acids, vitamins, lysine, cysteine, methionine, or threonine.

42. A vector comprising the modified nucleotide sequence of claim 36.

43. A host cell comprising the modified nucleotide sequence of claim 36 or a vector comprising the modified nucleotide sequence.

44. A method for producing fine chemicals comprising utilizing the modified nucleotide sequence of claim 36, a vector comprising the nucleotide sequence, or a host cell comprising the nucleotide sequence or vector for producing fine chemicals.

45. The method of claim 44, wherein the fine chemicals comprise amino acids, sugars, lipids, oils, fatty acids, vitamins, lysine, cysteine, methionine, or threonine.


SEQUENCE LISTING (22 sequences)

SEQ ID NO: 1 - DNA, 2217 bp, Corynebacterium glutamicum
atggctaagatcatctggacccgcaccgacgaagcaccgctgctcgcgacctactcgctgaagccggtcgtcgaggcatttgctgctaccgcgggcattgaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccaagaccaacgttgcaaccatggatgcaaacgacttccgccacaacgagaagtccatcatcctcgacgctgctgatgaagttcagatcaagcacatcgcagctgacggcaccgagaccatcctcaaggacagcctcaagcttcttgaaggcgaagttctagacggaaccgttctgtccgcaaaggcactggacgcattccttctcgagcaggtcgctcgcgcaaaggcagaaggtatcctcttctccgcacacctgaaggccaccatgatgaaggtctccgacccaatcatcttcggccacgttgtgcgcgcttacttcgcagacgttttcgcacagtacggtgagcagctgctcgcagctggcctcaacggcgaaaacggcctcgctgcaatcctctccggcttggagtccctggacaacggcgaagaaatcaaggctgcattcgagaagggcttggaagacggcccagacctggccatggttaactccgctcgcggcatcaccaacctgcatgtcccttccgatgtcatcgtggacgcttccatgccagcaatgattcgtacctccggccacatgtggaacaaagacgaccaggagcaggacaccctggcaatcatcccagactcctcctacgctggcgtctaccagaccgttatcgaagactgccgcaagaacggcgcattcgatccaaccaccatgggtaccgtccctaacgttggtctgatggctcagaaggctgaagagtacggctcccatgacaagaccttccgcatcgaagcagacggtgtggttcaggttgtttcctccaacggcgacgttctcatcgagcacgacgttgaggcaaatgacatctggcgtgcatgccaggtcaaggatgccccaatccaggattgggtaaagcttgctgtcacccgctcccgtctctccggaatgcctgcagtgttctggttggatccagagcgcgcacacgaccgcaacctggcttccctcgttgagaagtacctggctgaccacgacaccgagggcctggacatccagatcctctcccctgttgaggcaacccagctctccatcgaccgcatccgccgtggcgaggacaccatctctgtcaccggtaacgttctgcgtgactacaacaccgacctcttcccaatcctggagctgggcacctctgcaaagatgctgtctgtcgttcctttgatggctggcggcggactgttcgagaccggtgctggtggatctgctcctaagcacgtccagcaggttcaggaagaaaaccacctgcgttgggattccctcggtgagttcctcgcactggctgagtccttccgccacgagctcaacaacaacggcaacaccaaggccggcgttctggctgacgctctggacaaggcaactgagaagctgctgaacgaagagaagtccccatcccgcaaggttggcgagatcgacaaccgtggctcccacttctggctgaccaagttctgggctgacgagctcgctgctcagaccgaggacgcagatctggctgctaccttcgcaccagtcgcagaagcactgaacacaggcgctgcagacatcgatgctgcactgctcgcagttcagggtggagcaactgaccttggtggctactactcccctaacgaggagaagctcaccaacatcatgcgcccagtcgcacagttcaacgagatcgttgacgcactgaagaagtaa

SEQ ID NO: 2 - DNA, 2217 bp, Artificial Sequence
  Isocitrate Dehydrogenase, Corynebacterium glutamicum, ATG-GTG mutation in the first codon
gtggctaagatcatctggacccgcaccgacgaagcaccgctgctcgcgacctactcgctgaagccggtcgtcgaggcatttgctgctaccgcgggcattgaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccaagaccaacgttgcaaccatggatgcaaacgacttccgccacaacgagaagtccatcatcctcgacgctgctgatgaagttcagatcaagcacatcgcagctgacggcaccgagaccatcctcaaggacagcctcaagcttcttgaaggcgaagttctagacggaaccgttctgtccgcaaaggcactggacgcattccttctcgagcaggtcgctcgcgcaaaggcagaaggtatcctcttctccgcacacctgaaggccaccatgatgaaggtctccgacccaatcatcttcggccacgttgtgcgcgcttacttcgcagacgttttcgcacagtacggtgagcagctgctcgcagctggcctcaacggcgaaaacggcctcgctgcaatcctctccggcttggagtccctggacaacggcgaagaaatcaaggctgcattcgagaagggcttggaagacggcccagacctggccatggttaactccgctcgcggcatcaccaacctgcatgtcccttccgatgtcatcgtggacgcttccatgccagcaatgattcgtacctccggccacatgtggaacaaagacgaccaggagcaggacaccctggcaatcatcccagactcctcctacgctggcgtctaccagaccgttatcgaagactgccgcaagaacggcgcattcgatccaaccaccatgggtaccgtccctaacgttggtctgatggctcagaaggctgaagagtacggctcccatgacaagaccttccgcatcgaagcagacggtgtggttcaggttgtttcctccaacggcgacgttctcatcgagcacgacgttgaggcaaatgacatctggcgtgcatgccaggtcaaggatgccccaatccaggattgggtaaagcttgctgtcacccgctcccgtctctccggaatgcctgcagtgttctggttggatccagagcgcgcacacgaccgcaacctggcttccctcgttgagaagtacctggctgaccacgacaccgagggcctggacatccagatcctctcccctgttgaggcaacccagctctccatcgaccgcatccgccgtggcgaggacaccatctctgtcaccggtaacgttctgcgtgactacaacaccgacctcttcccaatcctggagctgggcacctctgcaaagatgctgtctgtcgttcctttgatggctggcggcggactgttcgagaccggtgctggtggatctgctcctaagcacgtccagcaggttcaggaagaaaaccacctgcgttgggattccctcggtgagttcctcgcactggctgagtccttccgccacgagctcaacaacaacggcaacaccaaggccggcgttctggctgacgctctggacaaggcaactgagaagctgctgaacgaagagaagtccccatcccgcaaggttggcgagatcgacaaccgtggctcccacttctggctgaccaagttctgggctgacgagctcgctgctcagaccgaggacgcagatctggctgctaccttcgcaccagtcgcagaagcactgaacacaggcgctgcagacatcgatgctgcactgctcgcagttcagggtggagcaactgaccttggtggctactactcccctaacgaggagaagctcaccaacatcatgcgcccagtcgcacagttcaacgagatcgttgacgcactgaagaagtaa

SEQ ID NO: 3 - DNA, 1002 bp, Artificial Sequence
  Vectorinsert with Isocitrate Dehydrogenase, Corynebacterium glutamicum, ATG-GTG Mutation
ctcgagcgaagacctcgcagattccgatattccaggaaccgccatgatcgaaatcccctcagatgacgatgcacttgccatcgagggaccttcctccatcgatgtgaaatggctgccccgcaacggccgcaagcacggtgaattgttgatggaaaccctggccctccaccatgaagaaacagaagctgcagccacctccgaaggcgaacttgtgtgggagactcctgtgttctccgccactggcgaacagatcacagaatccaacccacgttcaggcgactactactggattgctggcgaaagtggtgtcgtgaccagcattcgtcgatctctagtgaaagagaaaggcctcgaccgttcccaagtggcattcatggggtattggaaacacggcgtttccatgcggggctgaaactgccaccataggcgccagcaattagtagaacactgtattctaggtagctgaacaaaagagcccatcaaccaaggagactcgtggctaagatcatctggacccgcaccgacgaagcaccgctgctcgcgacctactcgctgaagccggtcgtcgaggcatttgctgctaccgcgggcattgaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccacgcgt

SEQ ID NO: 4 - DNA, 2217 bp, Artificial Sequence
  Isocitrate Dehydrogenase, Corynebacterium glutamicum, GGC ATT--->GGG ATA mutation in positions 32 and 33
atggctaagatcatctggacccgcaccgacgaagcaccgctgctcgcgacctactcgctgaagccggtcgtcgaggcatttgctgctaccgcggggatagaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccaagaccaacgttgcaaccatggatgcaaacgacttccgccacaacgagaagtccatcatcctcgacgctgctgatgaagttcagatcaagcacatcgcagctgacggcaccgagaccatcctcaaggacagcctcaagcttcttgaaggcgaagttctagacggaaccgttctgtccgcaaaggcactggacgcattccttctcgagcaggtcgctcgcgcaaaggcagaaggtatcctcttctccgcacacctgaaggccaccatgatgaaggtctccgacccaatcatcttcggccacgttgtgcgcgcttacttcgcagacgttttcgcacagtacggtgagcagctgctcgcagctggcctcaacggcgaaaacggcctcgctgcaatcctctccggcttggagtccctggacaacggcgaagaaatcaaggctgcattcgagaagggcttggaagacggcccagacctggccatggttaactccgctcgcggcatcaccaacctgcatgtcccttccgatgtcatcgtggacgcttccatgccagcaatgattcgtacctccggccacatgtggaacaaagacgaccaggagcaggacaccctggcaatcatcccagactcctcctacgctggcgtctaccagaccgttatcgaagactgccgcaagaacggcgcattcgatccaaccaccatgggtaccgtccctaacgttggtctgatggctcagaaggctgaagagtacggctcccatgacaagaccttccgcatcgaagcagacggtgtggttcaggttgtttcctccaacggcgacgttctcatcgagcacgacgttgaggcaaatgacatctggcgtgcatgccaggtcaaggatgccccaatccaggattgggtaaagcttgctgtcacccgctcccgtctctccggaatgcctgcagtgttctggttggatccagagcgcgcacacgaccgcaacctggcttccctcgttgagaagtacctggctgaccacgacaccgagggcctggacatccagatcctctcccctgttgaggcaacccagctctccatcgaccgcatccgccgtggcgaggacaccatctctgtcaccggtaacgttctgcgtgactacaacaccgacctcttcccaatcctggagctgggcacctctgcaaagatgctgtctgtcgttcctttgatggctggcggcggactgttcgagaccggtgctggtggatctgctcctaagcacgtccagcaggttcaggaagaaaaccacctgcgttgggattccctcggtgagttcctcgcactggctgagtccttccgccacgagctcaacaacaacggcaacaccaaggccggcgttctggctgacgctctggacaaggcaactgagaagctgctgaacgaagagaagtccccatcccgcaaggttggcgagatcgacaaccgtggctcccacttctggctgaccaagttctgggctgacgagctcgctgctcagaccgaggacgcagatctggctgctaccttcgcaccagtcgcagaagcactgaacacaggcgctgcagacatcgatgctgcactgctcgcagttcagggtggagcaactgaccttggtggctactactcccctaacgaggagaagctcaccaacatcatgcgcccagtcgcacagttcaacgagatcgttgacgcactgaagaagtaa

SEQ ID NO: 5 - DNA, 1002 bp, Artificial Sequence
  Vectorinsert with Isocitrate Dehydrogenase, Corynebacterium glutamicum, GGC ATT--->GGG ATA mutation in positions 32 and 33
ctcgagcgaagacctcgcagattccgatattccaggaaccgccatgatcgaaatcccctcagatgacgatgcacttgccatcgagggaccttcctccatcgatgtgaaatggctgccccgcaacggccgcaagcacggtgaattgttgatggaaaccctggccctccaccatgaagaaacagaagctgcagccacctccgaaggcgaacttgtgtgggagactcctgtgttctccgccactggcgaacagatcacagaatccaacccacgttcaggcgactactactggattgctggcgaaagtggtgtcgtgaccagcattcgtcgatctctagtgaaagagaaaggcctcgaccgttcccaagtggcattcatggggtattggaaacacggcgtttccatgcggggctgaaactgccaccataggcgccagcaattagtagaacactgtattctaggtagctgaacaaaagagcccatcaaccaaggagactcatggctaagatcatctggacccgcaccgacgaagcaccgctgctcgcgacctactcgctgaagccggtcgtcgaggcatttgctgctaccgcggggatagaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccacgcgt

SEQ ID NO: 6 - DNA, 32 bp, Artificial Sequence
  Oligonucleotide; Primer old 441
gagtacctcgagcgaagacctcgcagattccg

SEQ ID NO: 7 - DNA, 32 bp, Artificial Sequence
  Oligonucleotide; Primer old 442
catgagacgcgtggaatctgcagaccactcgc

SEQ ID NO: 8 - DNA, 24 bp, Artificial Sequence
  Oligonucleotide; Primer old 443
gagactcgtggctaagatcatctg

SEQ ID NO: 9 - DNA, 24 bp, Artificial Sequence
  Oligonucleotide; Primer old 444
cagatgatcttagccacgagtctc

SEQ ID NO: 10 - DNA, 18 bp, Artificial Sequence
  Oligonucleotide; Primer old 447
ctaccgcggggatagagg

SEQ ID NO: 11 - DNA, 18 bp, Artificial Sequence
  Oligonucleotide; Primer Old 448
cctctatccccgcggtag

SEQ ID NO: 12 - DNA, 20 bp, Artificial Sequence
  Oligonucleotide; Primer BK1776
aacggcaggtatatgtgatg

SEQ ID NO: 13 - DNA, 18 bp, Artificial Sequence
  Oligonucleotide; Primer Old 450
cgagtaggtcgcgagcag

SEQ ID NO: 14 - DNA, 21 bp, Artificial Sequence
  Oligonucleotide; Primer Old 471
gaatccaacccacgttcaggc

SEQ ID NO: 15 - DNA, 5293 bp, Artificial Sequence
  plasmid pClik int sacB ICDH (ATG-GTG)
ctcgagcgaagacctcgcagattccgatattccaggaaccgccatgatcgaaatcccctcagatgacgatgcacttgccatcgagggaccttcctccatcgatgtgaaatggctgccccgcaacggccgcaagcacggtgaattgttgatggaaaccctggccctccaccatgaagaaacagaagctgcagccacctccgaaggcgaacttgtgtgggagactcctgtgttctccgccactggcgaacagatcacagaatccaacccacgttcaggcgactactactggattgctggcgaaagtggtgtcgtgaccagcattcgtcgatctctagtgaaagagaaaggcctcgaccgttcccaagtggcattcatggggtattggaaacacggcgtttccatgcggggctgaaactgccaccataggcgccagcaattagtagaacactgtattctaggtagctgaacaaaagagcccatcaaccaaggagactcgtggctaagatcatctggacccgcaccgacgaagcaccgctgctcgcgacctactcgctgaagccggtcgtcgaggcatttgctgctaccgcgggcattgaggtcgagacccgggacatttcactcgctggacgcatcctcgcccagttcccagagcgcctcaccgaagatcagaaggtaggcaacgcactcgcagaactcggcgagcttgctaagactcctgaagcaaacatcattaagcttccaaacatctccgcttctgttccacagctcaaggctgctattaaggaactgcaggaccagggctacgacatcccagaactgcctgataacgccaccaccgacgaggaaaaagacatcctcgcacgctacaacgctgttaagggttccgctgtgaacccagtgctgcgtgaaggcaactctgaccgccgcgcaccaatcgctgtcaagaactttgttaagaagttcccacaccgcatgggcgagtggtctgcagattccacgcgtcatatgactagttcggacctagggatatcgtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaat

SEQ ID NO: 16 - DNA, 906 bp, Corynebacterium glutamicum
atgagcacaggtttaacagctaagaccggagtagagcacttcggcaccgttggagtagcaatggttactccattcacggaatccggagacatcgatatcgctgctggccgcgaagtcgcggcttatttggttgataagggcttggattctttggttctcgcgggcaccactggtgaatccccaacgacaaccgccgctgaaaaactagaactgctcaaggccgttcgtgaggaagttggggatcgggcgaagctcatcgccggtgtcggaaccaacaacacgcggacatctgtggaacttgcggaagctgctgcttctgctggcgcagacggccttttagttgtaactccttattactccaagccgagccaagagggattgctggcgcacttcggtgcaattgctgcagcaacagaggttccaatttgtctctatgacattcctggtcggtcaggtattccaattgagtctgataccatgagacgcctgagtgaattacctacgattttggcggtcaaggacgccaagggtgacctcgttgcagccacgtcattgatcaaagaaacgggacttgcctggtattcaggcgatgacccactaaaccttgtttggcttgctttgggcggatcaggtttcatttccgtaattggacatgcagcccccacagcattacgtgagttgtacacaagcttcgaggaaggcgacctcgtccgtgcgcgggaaatcaacgccaaactatcaccgctggtagctgcccaaggtcgcttgggtggagtcagcttggcaaaagctgctctgcgtctgcagggcatcaacgtaggagatcctcgacttccaattatggctccaaatgagcaggaacttgaggctctccgagaagacatgaaaaaagctggagttctataa

SEQ ID NO: 17 - DNA, 906 bp, Artificial Sequence
  optimized dapA of Corynebacterium glutamicum
atgagcacagggttaacagctaagacaggggtagagcacttcgggaccgttggggtagcaatggttactccattcacggaatccggagacatcgatatcgctgctggccgcgaagtcgcggcttatttggttgataagggcttggattctttggttctcgcgggcaccactggtgaatccccaacgacaaccgccgctgaaaaactagaactgctcaaggccgttcgtgaggaagttggggatcgggcgaagctcatcgccggtgtcggaaccaacaacacgcggacatctgtggaacttgcggaagctgctgcttctgctggcgcagacggccttttagttgtaactccttattactccaagccgagccaagagggattgctggcgcacttcggtgcaattgctgcagcaacagaggttccaatttgtctctatgacattcctggtcggtcaggtattccaattgagtctgataccatgagacgcctgagtgaattacctacgattttggcggtcaaggacgccaagggtgacctcgttgcagccacgtcattgatcaaagaaacgggacttgcctggtattcaggcgatgacccactaaaccttgtttggcttgctttgggcggatcaggtttcatttccgtaattggacatgcagcccccacagcattacgtgagttgtacacaagcttcgaggaaggcgacctcgtccgtgcgcgggaaatcaacgccaaactatcaccgctggtagctgcccaaggtcgcttgggtggagtcagcttggcaaaagctgctctgcgtctgcagggcatcaacgtaggagatcctcgacttccaattatggctccaaatgagcaggaacttgaggctctccgagaagacatgaaaaaagctggagttctataa

SEQ ID NO: 18 - DNA, 8438 bp, Artificial Sequence
  plasmid pK19 PSOD ask
aattcgcccttcaccgcggctttggacatcactgctacgtagccaaacaatgcacccgtcacaagaccaaggatgagggctttgtccttctttaatacgtattccgcaagcagccacattccacccattactgcaacgccgactaaaagtactggaatccatcgatcgagtgggggtgggggtttccgggaagggggcgtcccaaaacgatcatgatgcccacggctacggtgaggagggtagcccagaagatttcagttcggcgtagtcggtagccattgaatcgtgctgagagcggcagcgtgaacatcagcgacaggacaagcactggttgcactaccaagagggtgccgaaaccaagtgctactgtttgtaagaaatatgccagcatcgcggtactcatgcctgcccaccacatcggtgtcatcagagcattgagtaaaggtgagctccttagggagccatcttttggggtgcggagcgcgatccggtgtctgaccacggtgccccatgcgattgttaatgccgatgctagggcgaaaagcacggcgagcagattgctttgcacttgattcagggtagttgactaaagagttgctcgcgaagtagcacctgtcacttttgtctcaaatattaaatcgaatatcaatatatggtctgtttattggaacgcgtcccagtggctgagacgcatccgctaaagccccaggaagggcgaattctgcagatatccatcacactggcggccgctcgagcatgcatctagcttatcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattccgtggcacggaaatcgaggtagaagacattactcaggcaaccgaaagggcgaattccgtggcacggaaatcgaggtagaagacattactcaggcaaccgaaagggcgaattcgcccttgcggcgcaggattttctaaaacaggatgcctgccacctttaagcgcctcatcagcggtaaccatcacgggttcgggtgcgaaaaaccatgccataacaggaatgttcctttcgaaaattgaggaagccttatgcccttcaaccctacttagctgccaattattccgggcttgtgacccgctacccgataaataggtcggctgaaaaatttcgttgcaatataaacaaaaaggcctatcattgggaggtgtcgcaccaagtacttttgcgaagcgccatctgacggattttcaaaagatgtatatgctcggtgcggaaacctacgaaaggattttttacccgtggccctggtcgtacagaaatatggcggttcctcgcttgagagtgcggaacgcattagaaacgtcgctgaacggatcgttgccaccaagaaggctggaaatgatgtcgtggttgtctgctccgcaatgggagacaccacggatgaacttctagaacttgcagcggcagtgaatcccgttccgccagctcgtgaaatggatatgctcctgactgctggtgagcgtatttctaacgctctcgtcgccatggctattgagtcccttgcgcagaagcccaatctttcacgggctctcaggctggtgtgctcaccaccgagcgccacggaaacgcacgcattgttgatgtcactccaggtcgtgtgcgtgaagcactcgatgagggcaagatctgcattgttgctggtttccagggtgttaataaagaaacccgcgatgtcaccacgttgggtcgtggtggttctgacaccactgcagttgcgttggcagctgctttgaacgctgatgtgtgtgagatttactcggacgttgacggtgtgtataccgctgacccgcgcatcgttcctaatgcacagaagctggaaaagctcagcttcgaagaaatgctggaacttgctgctgttggctccaagattttggtgctgcgcagtgttgaatacgctcgtgcattcaatgtgccacttcgcgtacgctcgtcttatagtaatgatcccggcactttgattgccggctctatggaggatattcctgtggaagaagcagtccttaccggtgtcgcaaccgacaagtccgaagccaaagtaaccgttctgggtatttccgataagccaggcgaggctgcgaaggttttccgtgcgttggctgatgcagaaatcaacattgacatggttctgcagaacgtctcttctgtagaagacggcaccaccgacatcatcttcacctgccctcgttccgacggccgccgcgcgatggagatcttgaagaagcttcaggttcagggcaactggaccaatgtgctttacgacgaccaggtcggcaaagtctccctcgtgggtgctggcatgaagtctcacccaggtgttaccgcagagttcatggaagctctgcgcgatgtcaacgtgaacatcgaattgatttccacctctgagattcgtatttccgtgctgatccgtgaagatgatctggatgctgctgcacgtgcattgcatgagcagttccagctgggcggcgaagacgaagccgtcgtttatgcaggcaccggacgctaaagttttaaaggagtagttttacaatgaccaccatcgcagttgttggtgcaaccggccaggtcggccaggttatgcgcacccttttggaagagcgcaatttcccagctgacactgttcgtttctttgcttccccacgttccgcaggccgtaagattgaattcgccctttcggttgcctgagtaatgtcttctacctcgatttccgtgccacggaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagggcgtcccggaaaacgattccgaagcccaacctttcatagaaggcggcggtggaatcgaaatctcgtgatggcaggttgggcgtcgcttggtcggtcatttcgctcggtacccatcggcattttcttttgcgtttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagtaacggccgccagtgtgctgg

SEQ ID NO: 19 - DNA, 5685 bp, Artificial Sequence
  plasmid pCLIK Int Psod Glucose-6-PDH
cgcgtttgcccaaatagtggtcgatgcggaacacagaagattctgggaagactgcgttgaccagctggttgagctcgtgtgcggattcgaggttgtggccgaaaggcttctcgatgatcacgcggcgccatgcttcttcggtggattcagccatgccggaacgctccagctggtggcagaccgctgtgaaggaatctggtggaatggacaggtagtaagcccagttgccggcggtgccgcgggttttgtcgatgcgcttgagtgttgcagcgaggttgtcgaaagctgcatcatcatcaaagttgccgcgaacaaattccataccctcggcgaggcgctcccaaacattttcacggaattccgtacgagcaccagcacttgcggcatcgcgtacgtatttttcaaagtcttctttggaccattcgcggcggccgtaacctaccaacgagaatcctgggggcagcaatccgcggtttgctagatcataaatggcggggagcagcttctttcgagccaagtcgccagtgacaccgaagatcaccatgccggaagggccagcgatgcgggggagtcgtttatcctgcgggtcgcgcagtgggtttgtccagctggagggggtcgtgtttgtgctcacgggtaaaaaatcctttcgtaggtttccgcaccgagcatatacatcttttgaaaatccgtcagatggcgcttcgcaaaagtacttggtgcgacacctcccaatgataggcctttttgttgatattgcaacgaaatttttcagccgacctatttatcgggtagcgggtcacaagcccggaataattggcagctagatggtagtgtcacgatcctttctttaatgaaagatgtgtaacggccacataagatcgaactaattcgatttcatgtcgccgttactgatgcagcgtgctgattctacttcagacgagcttccatggactcaagcagttcgctccaagaagcaacgaacttgtccacaccctcggtctccaggacctggaagacatctgccaagtcaacgcccagagcctcaagctgggagaacacagcgtcagcttctgccgcggagttggacagggtgtcaccgtgcaggttgccctgctccagaaccgcgtcgatggtgccttctggcatggtgttgacggtgtttggaccagccagctcggaaacgtaaagagttgcagcgtacgcagggttcttcacgccggtggatgcccacagtgggcgctgagtgttggcaccttcaggcagctcggcggcgtcgaaaagctccttgtacacagcgtaagcgcgctgagcgttggcaacgcctgccttgccgcgcagagccaaagcctcatcggatccgattgcctcgaggcgcttgtcgatctcaacgtcgacatcgatgctcttctgcgttaattaacaattgggatcctctagacccgggatttaaatgatccgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtacca

SEQ ID NO: 20 - DNA, 7632 bp, Artificial Sequence
  plasmid pK19 sacB Glu-6-P-DH
cgcccttcccaacagttgcgcagcctgaatggcgaatggcgataagctagcttcacgctgccgcaagcactcagggcgcaagggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcggatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgctagaggatcgatcctttttaacccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagtttctgaatttgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccgatgggtaccgagcgaaatgaccgaccaagcgacgcccaacctgccatcacgagatttcgattccaccgccgccttctatgaaaggttgggcttcggaatcgttttccgggacgccctcgcggacgtgctcatagtccacgacgcccgtgattttgtagccctggccgacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagggctggatgatcctccagcgcggggatctcatgctggagttcttcgcccaccccaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgaaatttgctgggtggtggtatccggaagttcaaagatcattttttgcccctaaattatggcctgcgccaggtgtgaccgttgcgggaaagcatttcatcagcgctctttggaccccacgtacccgctgggtaatcctctggttctccatcggcatcccatgcttcaagaattggatccagaatcttccagctcagttccacttcctcgttggtagggaagaggctggattcatctaacagcgcatccaaaatgaggcgctcgtatgcttcaggtgattcttcagtgaaggattctgagtaggagaagtccatgttgacgtcacggacttccatggcagaacctggaaccttggaaccgaagcggatgagcacaccttcatcaggctgcacgcgaatcacgatggcgttttggccaagggatacagtcatgtcgccgtcgaaaggctggtgtggtgcgtctttaaacaccacggcaatctcagtaacacggcgaccaagacgcttaccggtgcgcaggtagaacggcacaccagcccagcgacgagacgtgatctctaaggtacaagccgcaaaagtctcagtggtggactcagggttgaagccatcttcttcgcgaagtcccttgactaactcagagccctgccaaccggcagcgtactgaccacgagcggaggttttatccaatgggtagcacggctttgtcgcagagagcaccttgatcttttctgcctgcagctgcgctggcacgaaagaaattggttcttccatggcaaccagagccaagagctggatcaggtggttctggatgacgtcgcgggctgcgccgatgccgtcgtagtaaccagcacgtccacccaagccaatatcttcagtcatggtgatctggacgtggtcaacgtagttggagttccacagtggctcaaacagctggttagcaaaacgcagagccaggatgttttgaactgtttccttgcccaaatagtggtcgatgcggaacacagaagattctgggaagactgcgttgaccagctggttgagctcgtgtgcggattcgaggttgtggccgaaaggcttctcgatgatcacgcggcgccatgcttcttcggtggattcagccatgccggaacgctccagctggtggcagaccgctgtgaaggaatctggtggaatggacaggtagtaagcccagttgccggcggtgccgcgggttttgtcgatgcgcttgagtgttgcagcgaggttgtcgaaagctgcatcatcatcaaagttgccgcgaacaaattccataccctcggcgaggcgctcccaaacattttcacggaattccgtacgagcaccagcacttgcggcatcgcgtacgtatttttcaaagtcttctttggaccattcgcggcggccgtaacctaccaacgagaatcctgggggcagcaatccgcggtttgctagatcataaatggcggggagcagcttctttcgagccaagtcgccagtgacaccgaagatcaccatgccggaagggccagcgatgcgggggagtcgtttatcctgcgggtcgcgcagtgggtttgtccagctggagggggtcgtgtttgtgctcacgatggtagtgtcacgatcctttctttaatgaaagatgtgtaacggccacataagatcgaactaattcgatttcatgtcgccgttactgatgcagcgtgctgattctacttcagacgagcttccatggactcaagcagttcgctccaagaagcaacgaacttgtccacaccctcggtctccaggacctggaagacatctgccaagtcaacgcccagagcctcaagctgggagaacacagcgtcagcttctgccgcggagttggacagggtgtcaccgtgcaggttgccctgctccagaaccgcgtcgatggtgccttctggcatggtgttgacggtgtttggaccagccagctcggaaacgtaaagagttgcagcgtacgcagggttcttcacgccggtggatgcccacagtgggcgctgagtgttggcaccttcaggcagctcggcggcgtcgaaaagctccttgtacacaggaattccgg

SEQ ID NO: 21 - DNA, 7438 bp, Artificial Sequence
  plasmid pK19 PGro PycA
ttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcaggaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcgatcctctagcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagatgcatgctcgagcggccgccagtgtgatggatatctgcagaattcgcccttccggcgaagtgtctgctcgcgtgattgtgcttcctttggctactaacccacgcgccaagatgcgttccctgcgccacggttttgtgaagctgttctgccgccgtaactctggcctgatcatcggtggtgtcgtggtggcaccgaccgcgtctgagctgatcctaccgatcgctgtggcagtgaccaaccgtctgacagttgctgatctggctgataccttcgcggtgtacccatcattgtcaggttcgattactgaagcagcacgtcagctggttcaacatgatgatctaggctaatttttctgagtcttagattttgagaaaacccaggattgctttgtgcactcctgggttttcactttgttaagcagttttggggaaaagtgcaaagtttgcaaagtttagaaatattttaagaggtaagatgtctgcaggtggaagcgtttaaatgcgttaaacttggccaaatgtggcaacctttgcaaggtgaaaaactggggcggggtaagggcgaattccagcacactggcggccgttactagcttatcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcaacctgtggcgcaacgctgtatataacctgcgtacggcttaaagtttggctgccatgtgaatttttagcaccctcaacagttgagtgctggcactctcgggggtagagtgccaaataggttgtttgacacacagttgttcacccgcgacgacggctgtgctggaaacccacaaccggcacacacaaaatttttctcatggagggattcatcgtgtcgactcacacatcttcaacgcttccagcattcaaaaagatcttggtagcaaaccgcggcgaaatcgcggtccgtgctttccgtgcagcactcgaaaccggtgcagccacggtagctatttacccccgtgaagatcggggatcattccaccgctcttttgcttctgaagctgtccgcattggtaccgaaggctcaccagtcaaggcgtacctggacatcgatgaaattatcggtgcagctaaaaaagttaaagcagatgccatttacccgggatacggcttcctgtctgaaaatgcccagcttgcccgcgagtgtgcggaaaacggcattacttttattggcccaaccccagaggttcttgatctcaccggtgataagtctcgcgcggtaaccgccgcgaagaaggctggtctgccagttttggcggaatccaccccgagcaaaaacatcgatgagatcgttaaaagcgctgaaggccagacttaccccatctttgtgaaggcagttgccggtggtggcggacgcggtatgcgttttgttgcttcacctgatgagcttcgcaaattagcaacagaagcatctcgtgaagctgaagcggctttcggcgatggcgcggtatatgtcgaacgtgctgtgattaaccctcagcatattgaagtgcagatccttggcgatcacactggagaagttgtacacctttatgaacgtgactgctcactgcagcgtcgtcaccaaaaagttgtcgaaattgcgccagcacagcatttggatccagaactgcgtgatcgcatttgtgcggatgcagtaaagttctgccgctccattggttaccagggcgcgggaaccaagggcgaattcctctggataatcatcgcggtagttacgagcggcgcgaatgcaagggcgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagggcgtcccggaaaacgattccgaagcccaacctttcatagaaggcggcggtggaatcgaaatctcgtgatggcaggttgggcgtcgcttggtcggtcatttcgctcggtacccatcggcattttcttttgcgttt

SEQ ID NO: 22 - DNA, 4980 bp, Artificial Sequence
  plasmid pCLIK int sacB dapA codon low
acgcgtgcggtagaatgtttaagaaagctgcaggtagcagcgccaactgttttcggtgattttgagattgaaactttggcagacggatcgcaaatggcaacaagcccgtatgtcatggacttttaacgcaaagctcacacccacgagctaaaaattcatatagttaagacaacatttttggctgtaaaagacagccgtaaaaacctcttgctcgtgtcaattgttcttatcggaatgtggcttgggcgattgttatgcaaaagttgttaggttttttgcggggttgtttaacccccaaatgagggaagaaggtaaccttgaactctatgagcacagggttaacagctaagacaggggtagagcacttcgggaccgttggggtagcaatggttactccattcacggaatccggagacatcgatatcgctgctggccgcgaagtcgcggcttatttggttgataagggcttggattctttggttctcgcgggcaccactggtgaatccccaacgacaaccgccgctgaaaaactagaactgctcaaggccgttcgtgaggaagttggggatcgggcgaagctcatcgccggtgtcggaaccaacaacacgcggacatctgtggaacttgcggaagctgctgcttctgctggcgcagacggccttttagttgtaactccttattactccaagccggtcgacatcgatgctcttctgcgttaattaacaattgggatcctctagacccgggatttaaatcgctagcgggctgctaaaggaagcggaacacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgctagcggcgcgccggccggcccggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaaggccggccgcggccgccatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcggcggccgctcgatttaaatctcgagaggcctgacgtcgggcccggtacc